(19) United States
(12) Patent Application Publication
(10) Pub. No.: US 2006/0073212 A1
(43) Pub. Date: Apr. 6, 2006

(54) Method of treating respiratory disorders and airway inflammation

(76) Inventors: Craig R. Palmer, San Francisco, CA (US); Emil Y. Chi, Seattle, WA (US)

Correspondence Address: MAYER, BROWN, ROWE & MAW LLP, P.O. BOX 2828, CHICAGO, IL, 60690-2828, US

(21) Appl. No.: 11/112,561

(22) Filed: Apr. 22, 2005

Related U.S. Application Data
(60) Provisional application No. 60/564,318, filed on Apr. 22, 2004.
(60) Provisional application No. 60/572,363, filed on May 19, 2004.

(51) Int. Cl. A61K 33/14; A61K 33/00; A61K 33/40

(52) U.S. Cl. 424/600; 424/661; 424/616

(57) ABSTRACT

Compositions and methods for treating or preventing respiratory disorders and airway inflammation in an animal are disclosed. In one embodiment, the respiratory disorder is asthma. In one embodiment, the airway inflammation is asthma-related. In one embodiment, the respiratory disorder is cystic fibrosis. The method comprises administering an electrolyzed saline solution to the animal. In one embodiment, the electrolyzed saline solution comprises about 0.1 ppm to about 100 ppm ozone and one or more active species selected from the group consisting of: about 5 ppm to about 300 ppm of at least one active chlorine species, about 0.1 ppm to about 300 ppm of at least one active oxygen species, about 5 ppm to about 300 ppm active hydrogen species, and combinations thereof. A mouse model of cystic fibrosis and methods of screening pharmaceutical agents are also provided.

### RELATED APPLICATIONS

This application claims priority to U.S. Provisional Application Ser. No. 60/564,318 filed Apr. 22, 2004 and U.S. Provisional Patent Application No. 60/572,363 filed May 19, 2004, both of which are hereby incorporated by reference herein.

### BACKGROUND

Airway secretions and their constituents play an important part in the defense of the respiratory tract. Respiratory secretions typically consist of a mixture of mucus, glandular products and plasma proteins and are produced by submucosal glands, goblet cells, and mucous cells located through the tracheobronchial system. Airway mucus secretion in general is poorly understood. Alder, K. L., Y. Li., Am J Respir Cell Mol Bio, 25:397-400 (2001).

In some cases, respiratory mucus is overproduced. The overproduction can be due to over-expression of mucin genes, mucus cell hyperplasia and/or hypersecretion in the airway. Excess mucus can lead to obstruction of the airway, susceptibility to infection and a reduced defense against inflammatory airway diseases. Asthma, chronic bronchitis, and cystic fibrosis are diseases of excess mucus production in the airway.

Asthma is a disorder of the respiratory system characterized by airway hyperresponsiveness leading to acute and/or chronic airway inflammation. The severity of hyperreactivity typically correlates with the degree of inflammation. This inflammation of the bronchi and bronchioles reduces the airway space and can result in an asthma attack. Mucus hyper-secretion and mucus cell hyperplasia are seen in the lungs of patients with asthma. In chronic asthmatic individuals, mucus forms a viscoelastic gel, which is very sticky. When it stagnates, mucus can occlude the airway and become a favorable locus for bacteria or disease pathogenesis.

Cystic fibrosis (CF) is a hereditary disease characterized by excess mucus production, especially in the lungs, pancreas and intestines. Cystic fibrosis is one of the most common autosomal recessive disorders in the Caucasian population, affecting approximately 1 in 2,500 live births.

Cystic fibrosis patients are prone to lung infections. In fact, Pseudomonas aeruginosa colonizes almost all CF patients at some time during the disease process. Infection with Pseudomonas aeruginosa plays a major role in the pulmonary inflammation and injury associated with cystic fibrosis. There is increasing recognition that cystic fibrosis-associated lung injury and pulmonary inflammation may trigger a systemic inflammatory response, leading to widespread systemic effects on other organs. A number of P. aeruginosa components, including bacterium-associated products, such as alginate, and secreted products, have been described which contribute to P. aeruginosa-induced pathogenesis seen in cystic fibrosis patients. Starke, J. R., et al., Pediatr. Res., 22: 698-702 (1987); Heeckeren, A., et al., J. Clin. Invest., 100(11):2810-5 (1997).

Most CF patients are initially infected with classical, smooth strains of P. aeruginosa. The overproduction of the P. aeruginosa exopolysaccharide results in mucoid colony morphology, and is associated with the progressive deterioration of lung function in CF. Cripps, A. W., Immunol. Cell Biol., 73:418-24 (1995). The CF-associated mucus overproduction is believed to protect the bacteria from phagocytes and reactive oxygen species. Id.

Airway infection with P. aeruginosa triggers both acute and chronic inflammatory responses in CF. Starke, supra; Bonfield, T. L., Am. J. Respir. Crit. Care Med., 152:2111-8 (1995). Animal models of both acute and chronic lung infection have been used to study P. aeruginosa-induced airway inflammation, Pukhalsky, A. L., Mediators Inflamm., 8:159-67 (1999); Bonfield, supra. To date, however, no appropriate animal model of airway disease with profound and consistent over-secretion and over-production of mucus has been developed. Although P. aeurginosa can infect infant mice, Tang, H., et al., Infect. Immun., 63: 1278-1285 (1995), and some inflammatory responses are induced in mice when bacteria are administered in agar beads Heeckeren, A., et al., J. Clin. Invest., 100:2810-2815 (1997), there are no suitable in vivo models that reproduce the chronic colonization of the respiratory tract characteristic of patients with cystic fibrosis.

As such, there is a definite need for a therapy that effectively treats and/or prevents the incidence of respiratory disorders and airway inflammation, including asthma and cystic fibrosis, in mammals, such as animals and humans. Furthermore, there remains a need for an animal model of airway disease having characteristics of cystic fibrosis, including, e.g., over-secretion of mucus, and/or persistent colonization of the lungs upon infection with P. aeruginosa.

### SUMMARY OF THE INVENTION

Electrolyzed saline solutions suitable for the treatment or prevention of respiratory disorders and airway inflammation, including cystic fibrosis, asthma, and asthma-related airway inflammation, are disclosed. An animal model having characteristics of human cystic fibrosis, such as, e.g., over-secretion of mucus, is also disclosed.

In one embodiment, a method for treating or preventing a respiratory disorder in an animal, comprising administering an electrolyzed saline solution to the animal is provided. In one embodiment, the respiratory disorder comprises airway inflammation. In one embodiment, the respiratory disorder is asthma and the airway inflammation is asthma-related. In one embodiment, the respiratory disorder is cystic fibrosis.

In one embodiment, the electrolyzed saline solution comprises ozone and one or more active species selected from the group consisting of: active chlorine species, active oxygen species, and active hydrogen species, or combinations thereof. In one embodiment, the electrolyzed saline solution comprises ozone and at least one active chlorine species. In one embodiment, the electrolyzed saline solution comprises ozone, at least one active chlorine species, at least one active oxygen species, and at least one active hydrogen species. Exemplary active species include, e.g., HOCl⁻¹, OCl⁻¹, Cl⁻¹, Cl₂, O₂³, O₃, and H₂O₂.

The electrolyzed saline solution can comprise any amount of ozone and active species suitable for treating or preventing respiratory disorders and/or airway inflammation in an animal, such as, e.g., about 0.1 ppm to about 100 ppm ozone and about 5 ppm to about 300 ppm of at least one active chlorine species. The solution can also comprise about 0.1 ppm to about 300 ppm of at least one active oxygen species, and/or about 5 ppm to about 300 ppm of at least one active hydrogen species. In one embodiment, the solution comprises about 0.1 ppm to about 30 ppm ozone and about 10 ppm to about 100 ppm of at least one active chlorine species, and can also comprise about 0.1 ppm to about 100 ppm of at least one active oxygen species, and/or about 10 ppm to about 100 ppm of at least one active hydrogen species. In one embodiment, the solution comprises about 9 ppm to about 15 ppm ozone and about 10 ppm to about 80 ppm of at least one active chlorine species, and can also comprise about 0.1 ppm to about 80 ppm of at least one active oxygen species, and/or about 10 ppm to about 80 ppm of at least one active hydrogen species. In one embodiment, the electrolyzed saline solution comprises less than about 0.8 ppm ozone and about 10 ppm to about 80 ppm of at least one active chlorine species, such as, e.g., about 55 ppm to about 80 ppm of at least one active chlorine species. In yet another embodiment, the solution comprises about 0.30 to about 0.7 ppm ozone and about 10 ppm to about 80 ppm of at least one active chlorine species.

### BRIEF DESCRIPTION OF THE DRAWINGS

Many aspects of the invention can be better understood with reference to the following drawings:

FIG. 1A is a graph showing that administration of electrolyzed saline solution reduces the total cell influx in the bronchoalveolar lavage (“BAL”).

FIG. 1B is a graph showing that administration of electrolyzed saline solution reduces the percentage of eosinophils in the BAL.

FIG. 2A is a photograph showing BAL lavage fluids from an OVA-treated mouse challenged at days 25, 26, and 27.

FIG. 2B is a photograph showing BAL lavage fluids from a mouse treated with OVA and challenged and dosed with electrolyzed saline of the present invention (MDI-P).

FIG. 2C is a photograph showing BAL lavage fluids from the lungs of a mouse treated with saline only.

FIG. 3A is a photograph of H&E stained lung tissue from an OVA-immunized mouse that was challenged 3 times at days 25, 26, and 27.

FIG. 3B is a photograph of a PAS-stained lung tissue section tissue from an OVA-immunized mouse that was challenged 3 times at days 25, 26, and 27.

FIG. 4A is a photograph of lung tissue from a mouse treated with saline only.

FIG. 4B is a photograph of lung tissue from a mouse treated with MDI-P 50 without OVA immunization.

FIG. 5A is a photograph of lung tissue from mice that were OVA-sensitized/challenged and treated with MDI-P 100.

FIG. 5B is a photograph of lung tissue from mice that were OVA-sensitized/challenged and treated with MDI-P 100.

FIG. 6A is a photograph demonstrating histologic evidence of airway inflammation reduction in the lungs of mice treated with MDI-P 100.

FIG. 6B is a photograph demonstrating histologic evidence of airway inflammation reduction in the lungs of mice treated with MDI-P 100.

FIG. 6C is a photograph demonstrating histologic evidence of airway inflammation reduction in the lungs of mice treated with MDI-P 100.

FIG. 7A is a photograph showing lung tissue from OVA-treated mice.

FIG. 7B is a photograph showing lung tissue from MDI-P 50 and OVA immunized and challenged mice.

FIG. 8A is a photograph showing lung tissue from OVA sensitized/challenged mice.

FIG. 8B is a photograph showing lung tissue from OVA-sensitized/challenged mice treated with MDI-P 25.

FIG. 9A is a graph showing the level of cell infiltration in the lungs of each group.

FIG. 9B is a graph showing the % of EOS (eosinophils) in the lungs of each group.

FIG. 10A is a graph showing the level of mucus occlusion in each group.

FIG. 10B is a graph showing the percentage of mucus cells in each group.

FIG. 11A is a photograph of mouse lung treated with saline only. X80

FIG. 11B is a photograph of a mouse lung treated with MDI-P 100 only. X80

FIG. 11C is a photograph of an OVA immunized/challenged mouse lung. X80

FIG. 11D is a photograph of OVA immunized/challenged mouse lung after MDI-P100 treatment. X80

FIG. 12A is a photograph of a saline treated mouse that received 1×10⁶ P. aeruginosa through the nose for 30 minutes. X120

FIG. 12B is a photograph of a MDI-P 100 treated OVA immunized/challenged lung from a mouse that received 1×10⁶ P. aeruginosa through the nose for 30 minutes. X120

FIG. 12C is a photograph of an OVA-treated lung from a mouse that received 1×10⁶ P. aeruginosa through the nose for 30 minutes. X120

FIG. 13A is a photograph of a saline treated lung from a mouse 4 hours after bacteria incubation. X120

FIG. 13B is a photograph of a MDI-P100 treated lung from a mouse 4 hours after bacteria incubation. X120

FIG. 13C is a photograph of an OVA-treated lung from a mouse 4 hours after bacteria incubation. X120

FIG. 14A is a photograph of a saline treated mouse lung 24 hours after P. aeruginosa inoculation. X120

FIG. 14B is a photograph of a lung 24 hours after P. aeruginosa inoculation from an OVA immunized/challenged mouse inoculated with bacteria and treated with MDI-P100. X120

FIG. 14C is a photograph of a lung 24 hours after P. aeruginosa inoculation from an OVA treated and immunized mouse lung. X120

FIG. 15A is a photograph of a saline treated mouse lung 48 hours after P. aeruginosa inoculation. X120

FIG. 15B is a photograph of a lung 48 hours after P. aeruginosa inoculation from an OVA immunized/challenged mouse inoculated with bacteria and treated with MDI-P100. X120

FIG. 15C is a photograph of a lung 48 hours after P. aeruginosa inoculation from an OVA treated and immunized mouse lung. X120

FIG. 16A is a photograph of a lung from an OVA immunized/challenged mouse 48 hours after P. aeruginosa inoculation. X120

FIG. 16B is a photograph of a lung from an OVA immunized/challenged mouse 48 hours after P. aeruginosa inoculation. X180

FIG. 16B is a photograph of a lung from an OVA immunized/challenged mouse 48 hours after P. aeruginosa inoculation. X180

FIG. 17A is a photograph of a saline-treated mouse lung 24 hours after P. aeruginosa inoculation. X180

FIG. 17B is a photograph of an MDI-P 100 mouse lung 24 hours after P. aeruginosa inoculation. X180

FIG. 17C is a photograph of an OVA immunized/challenged mouse lung 24 hours after P. aeruginosa inoculation. X180

FIG. 18A is a photograph of a lung from an OVA immunized/challenged mouse 48 hours after inoculation with P. aeruginosa and treatment with MDI-P100.

FIG. 18B is a photograph of a lung from an OVA immunized/challenged mouse 48 hours after inoculation with P. aeruginosa.

FIG. 19A is a photograph of a lung from an OVA immunized/challenged mouse 48 hours after inoculation with P. aeruginosa. X180

FIG. 19B is a photograph of a lung from an OVA immunized/challenged mouse 48 hours after inoculation with P. aeruginosa. X180

FIG. 19C is a photograph of a lung from an OVA immunized/challenged mouse 48 hours after inoculation with P. aeruginosa. X180

FIG. 20A is a graph showing the level of mucus secretion, cell infiltration, and lung edema 0 hours after treatment.

FIG. 20B is a graph showing the amount of hemorrhage, PMN, and EOS 0 hours after treatment.

FIG. 21A is a graph showing the level of mucus secretion, cell infiltration, and lung edema 4 hours after treatment.

FIG. 21B is a graph showing the amount of hemorrhage, PMN, and EOS 4 hours after treatment.

FIG. 22A is a graph showing the level of mucus secretion, cell infiltration, and lung edema 24 hours after treatment.

FIG. 22B is a graph showing the amount of hemorrhage, PMN, and EOS 24 hours after treatment.

FIG. 23A is a graph showing the level of mucus secretion, cell infiltration, and lung edema 48 hours after treatment.

FIG. 23B is a graph showing the amount of hemorrhage, PMN, and EOS 48 hours after treatment.

### DETAILED DESCRIPTION OF THE INVENTION

It has now been found that saline solutions, which have been subjected to electrolysis to produce ozone and active products, such as, e.g., active chlorine products, active oxygen products, and active hydrogen products, are useful for the treatment and prevention of respiratory disorders and airway inflammation, such as asthma and cystic fibrosis, in an animal, such as a mammal, such as a human.

An animal model of airway disease having the characteristics of cystic fibrosis, such as, e.g., over-secretion of mucus, is also provided. Such animals provide a useful model for the over-secretion of mucus characteristic of cystic fibrosis pathogenesis.

Composition

The present invention provides electrolyzed saline solutions. In one embodiment, the electrolyzed saline solution comprises ozone and one or more active species selected from the group consisting of: active chlorine species, active oxygen species, and active hydrogen species, or combinations thereof. In one embodiment, the electrolyzed saline solution comprises ozone and at least one active chlorine species. In one embodiment, the electrolyzed saline solution comprises ozone, at least one active chlorine species, at least one active oxygen species, and at least one active hydrogen species. Exemplary active species include, e.g., HOCl⁻¹, OCL⁻¹, Cl⁻¹, Cl₂, O₂³, O₃, and H₂O₂.

The electrolyzed saline solution can comprise any amount of ozone and active species suitable for treating or preventing sepsis in an animal, such as, e.g., about 0.1 ppm to about 100 ppm ozone and about 5 ppm to about 300 ppm of at least one active chlorine species. The solution can also comprise about 0.1 ppm to about 300 ppm of at least one active oxygen species, and/or about 5 ppm to about 300 ppm of at least one active hydrogen species. In one embodiment, the solution comprises about 0.1 ppm to about 30 ppm ozone and about 10 ppm to about 100 ppm of at least one active chlorine species, and can also comprise about 0.1 ppm to about 100 ppm of at least one active oxygen species, and/or about 10 ppm to about 100 ppm of at least one active hydrogen species. In one embodiment, the solution comprises about 9 ppm to about 15 ppm ozone and about 10 ppm to about 80 ppm of at least one active chlorine species, and can also comprise about 0.1 ppm to about 80 ppm of at least one active oxygen species, and/or about 10 ppm to about 80 ppm of at least one active hydrogen species. In one embodiment, the electrolyzed saline solution comprises less than about 0.8 ppm ozone and about 10 ppm to about 80 ppm of at least one active chlorine species, such as, e.g., about 55 ppm to about 80 ppm of at least one active chlorine species. In yet another embodiment, the solution comprises about 0.30 to about 0.7 ppm ozone and about 10 ppm to about 80 ppm of at least one active chlorine species.

In one embodiment, the electrolyzed saline solution comprises about 5 ppm to about 80 ppm of at least one active hydrogen species, such as, e.g., about 10 ppm of at least one active hydrogen species. In one embodiment, the active hydrogen species is less than about 15 ppm. In one embodiment, the active hydrogen species is hydrogen peroxide.

In one embodiment, the electrolyzed saline solution comprises about 5 ppm to about 300 ppm of at least one active chlorine species, such as, e.g., free chlorine. In one embodiment, the electrolyzed saline solution comprises about 10 ppm to about 80 ppm of at least one active chlorine species. In one embodiment, the electrolyzed saline solution comprises about 55 ppm to about 80 ppm of at least one active chlorine species. In one embodiment, the electrolyzed saline solution comprises about 60 ppm of at least one active chlorine species. In one embodiment, the electrolyzed saline may or may not comprises ozone.

In one embodiment, the electrolyzed saline solution comprises about 5 ppm to about 300 ppm total chlorine. In one embodiment, the electrolyzed saline solution comprises about 10 ppm to about 80 ppm total chlorine, such as, e.g., about 50 ppm to about 70 ppm total chlorine, such as, e.g., about 60 ppm total chlorine.

In one embodiment, the electrolyzed saline solution has a redox potential of about 500 mV to about 1500 mV. In one embodiment, the electrolyzed saline solution has a redox potential of about 800 mV to about 900 mV, such as, e.g., about 850 mV.

In one embodiment, the concentration of active species can be expressed in terms of reactive oxygen species (ROS). In one embodiment, the electrolyzed saline solution comprises a sum of ROS activity from about 5 to about 100,000 μM relative to AAPH (2,2′-azobis(2-aminopropane) dihydrochloride) and/or allowing for inhibition of indicator strains within this range. In one embodiment, the electrolyzed saline solution has a ROS of from about 0.3 mM to about 10 mM ROS, such as, e.g., from about 0.5 mM to about 5 mM ROS, such as, e.g., from about 1 mM ROS to about 3 mM ROS, such as, e.g., from about 1 to about 2.5 mM ROS.

In one embodiment, the electrolyzed saline solution has an osmolarity from about 100 mOsm to about 500 mOsm, such as, e.g, about 200 mOsm to about 300 mOsm, such as, e.g., about 284 mOsm.

In one embodiment, the electrolyzed saline solution comprises less than about 4,000 ppm sodium, such as, e.g., about 3,900 ppm sodium.

The pH of the electrolyzed saline solution can be any pH suitable for the method of use. In one embodiment, the pH is from about 6.5 to about 8, such as, e.g., about 6.75 to about 7.5, about 7 to about 7.6, or about 7.2 to about 7.8. For example, in one embodiment the pH of the solution is in the range from about 7.2 to about 7.6. In one embodiment, the pH of the solution is in the range from about 6.75 to about 7.5. In one embodiment, when the solution is used for intravenous administration, the pH of the solution is in the range from about 7.35 to about 7.45 which is the pH range of human blood.

In one embodiment, the electrolyzed saline solution comprises less than about 0.8 ppm ozone and from about 55 ppm to about 80 ppm of at least one active chlorine species, such as, e.g., free chlorine. In one embodiment, the electrolyzed saline solution further comprises less than about 15 ppm active hydrogen species, such as, e.g., hydrogen peroxide. In one embodiment, the electrolyzed saline solution has a ROS from about 0.3 mM to about 10 mM, a pH from about 6.75 to about 7.5, and/or an osmolarity from about 200 mOsm to about 300 mOsm, such as, e.g., about 284 mOsm.

For purposes of this invention, the term “active species” or “active product” means any species or product resulting from the subjection of a saline solution to electrolysis, such as, e.g., an active chlorine species, an active oxygen species, and an active hydrogen species. The term “active species” or “active product” can mean one active species or a combination of active species. The term “active chlorine agent or species,” means one or more of any active form of chlorine resulting from the subjecting of a saline solution to electrolysis which can be measured by a chlorine selective electrode, such as, e.g., free chlorine, hypochlorous acid and the hypochlorite ion. The term “active oxygen agent or species” means one or more of any active form of oxygen resulting from the subjecting of a saline solution to electrolysis, such as, e.g., O₂³. The term “active hydrogen agent or species” means one or more of any active form of hydrogen resulting from the subjecting of a saline solution to electrolysis, such as, e.g., H₂O₂.

The composition can also comprise other products of the electrolysis reaction including ions selected from the group consisting of hydrogen, sodium and hydroxide ions. The interaction of the electrolysis products can result in a solution comprising bioactive atoms, radicals or ions selected from the group consisting of chlorine, ozone, hydroxide, hypochlorous acid, hypochlorite, peroxide, oxygen and perhaps others along with corresponding amounts of molecular hydrogen and sodium and hydrogen ions. In one embodiment, the electrolyzed saline solution comprises HOCl⁻¹, OCl⁻¹, Cl⁻¹, Cl₂, O₂³, O₃, and H₂O₂. The HOCl⁻¹, OCl⁻¹, Cl⁻¹, Cl₂, O₂³, O₃, and H₂O₂ can be present in any suitable amount, such as, e.g., about 0.1 ppm to about 300 ppm for each active species.

The electrolyzed saline solution can be prepared from any sterile saline solution suitable for producing the desired electrolyzed saline solution upon electrolysis. In one embodiment, the saline solution has an initial concentration from about 0.05% to about 10.0% NaCl. In one embodiment, the saline solution has an initial concentration from about 0.1% to about 5.0% NaCl. In another embodiment, the saline solution has an initial concentration from about 0.15% to about 1% NaCl, such as, e.g., an initial concentration from about 0.25% to about 1.0% NaCl. In one embodiment, the saline solution has an initial concentration of about 0.9% NaCl. In one embodiment, the saline solution has an initial concentration of about 0.45% NaCl. In one embodiment, the saline solution has an initial concentration of about 0.215% NaCl.

The saline solution can be subjected to electrolysis at any suitable voltage, current, and time to produce an appropriately electrolyzed solution. Suitable methods and equipment for performing the electrolysis are described in, e.g., U.S. Pat. Nos. 5,334,383; 5,507,932; 5,560,816; 5,622,848; 5,674,537; 5,731,008; 6,007,686; and 6,117,285, herein incorporated by reference. In one embodiment, the electrolysis reaction is performed at ambient temperatures.

In one embodiment, the saline solution is diluted with sterile distilled water to the desired concentration, such as, e.g., concentrations from about 0.05% to about 10.0% NaCl (e.g., about 0.1% to about 5.0% NaCl; about 0.15% to about 1% NaCl; or about 0.25% to about 1.0% NaCl). The diluted saline solution is then subjected to electrolysis at sufficient voltage, amperage and time to produce an electrolyzed solution comprising the desired concentrations of ozone and active chlorine, active oxygen, and/or active hydrogen species. The electrolysis reaction can be carried out at any suitable temperature. In one embodiment, the electrolysis reaction is carried out at ambient temperatures.

Obviously, the voltage and amperage to be used and the time of electrolysis is subject to many variables, i.e. the size and composition of the electrodes, the volume and/or concentration of saline being electrolyzed. For large electrodes or saline volumes or higher concentrations of saline solutions the voltage, amperage or time may be higher and/or longer. It is the generation of the desired concentration of ozone and active chlorine, active oxygen, and/or active hydrogen species that is important. According to Faraday's laws of electrolysis, the amount of chemical change produced by a current is proportional to the quantity of electricity passed. Also, the amounts of different substances liberated by a given quantity of electricity are proportional to the chemical equivalent weights of those substances.

Therefore, to generate an electrolyzed saline having the desired concentrations of ozone and active chlorine, active oxygen, and/or active hydrogen species from saline solutions having a saline concentration of less than about 1.0%, voltage, amperage and time parameters appropriate to the electrodes and solution are required to produce an electrolyzed solution comprising from about 0.1 to 100 ppm of ozone, such as, e.g., about less than 0.8 ppm ozone, and a free chlorine content from about 5 to 300 ppm, such as, e.g., about 55 ppm to about 80 ppm free chlorine. In one embodiment, the treatment produces an electrolyzed solution comprising from about 0.1 to about 50 ppm of ozone and a free chlorine content from about 10 to about 100 ppm. In a further embodiment, the treatment produces an electrolyzed solution comprising from about 0.1 to about 30 ppm of ozone and a free chlorine content from about 20 to about 60 ppm. In another embodiment, the treatment produces an electrolyzed solution comprising from about 0.1 to about 1.0 ppm ozone and a free chlorine content from about 50 to about 70 ppm. In yet another embodiment, the treatment produces an electrolyzed saline solution comprising less than about 0.8 ppm ozone and from about 55 to about 80 ppm of free chlorine.

The concentration of the active species can be measured by any suitable manner, such as, e.g., titration; methods described in Hoigne and Bader, Water Research, 5:449-456 (1981); HACH colorimeter Indigo method, or any other suitable method. Similarly, pH and redox potential can also be measured by any suitable method.

For in vitro use these solutions can be utilized without further modification or they can be adjusted as desired with saline or other solutions. For in vivo use these solutions can be utilized without further modification or they can be adjusted as desired with saline or other solutions. Prior to in vivo use, this solution may be adjusted or balanced to an isotonic saline concentration with sufficient hypertonic saline, e.g. 5% hypertonic saline solution.

In one embodiment, the electrolyzed saline solution is filtered prior to measurement. In one embodiment, the electrolyzed saline solution is filtered prior to administration or use.

In one embodiment, an electrolyzed saline solution can be obtained by subjecting about a 0.33% (about one third physiologically normal) saline solution to electrolysis for about 5 to 15 minutes. The voltage between the electrodes was maintained in the range of about 10 to 20 volts at a current in the range of about 5 to 20 amps, such that the freshly prepared electrolyzed saline when balanced or normalized with sterile 5% saline contained about 10 ppm to about 200 ppm of active chlorine species, such as, e.g., about 55 ppm to about 80 ppm of active chlorine species, along with about 0.1 to 30 ppm of ozone and corresponding amounts of molecular hydrogen and sodium and hydrogen ions.

In one embodiment, the electrolyzed saline solution remains stable in sealed sterile containers for a suitable period of time, such as, e.g., about 6 months, about one year or about 18 months.

Dosage and Dosage Forms

Particular dosages and methods of administration, as well as additional components to be administered, can be determined by those skilled in the art using the information set forth herein and set forth in the U.S. patent documents previously incorporated herein by reference.

An effective amount of the electrolyzed saline solution can be administered by any appropriate mode, e.g., intranasally, parenterally, e.g., intravenously (i.v.) or intraperitoneally (i.p.), orally, vaginally or rectally and may vary greatly according to the mode of administration, condition being treated, the size of the warm-blooded animal, etc. In one embodiment, the electrolyzed saline solution is administered in the form of an inhalant solution.

The electrolyzed saline solution of the present invention can be prepared in any suitable dosage form. In one embodiment, the electrolyzed saline solution can be formulated as a single pharmaceutical composition or as independent multiple pharmaceutical dosage forms. Pharmaceutical compositions according to the present invention include those suitable for intranasal, inhaled, topical, oral, rectal, buccal (for example, sublingual), or parenteral (for example, intravenous) administration, although the most suitable route in any given case will depend on the nature and severity of the condition being treated as well as by the type of mammal being treated. Of course, intravenous or intraperitoneal injections are typically suitable for delivering more active solution into the bloodstream of the animal. Such delivery can be suitable to quickly deliver the electrolyzed saline solution if the animal is suffering from a potentially fatal infection or disease state.

The electrolyzed saline solution of the present invention can be administered to any animal, such as, e.g., a mammal. In one embodiment, the mammal is a human. In one embodiment, the electrolyzed saline solution is used in a veterinary application for administration to mammals, reptiles, birds, exotic animals and farm animals, including, e.g., a monkey, or a lemur, a horse, a cow, a chicken, a pig, a dog, a cat, or a rodent, e.g., a rat, a mouse, a squirrel or a guinea pig. In one embodiment, the mammal is a food animal, such as any animal suitable for serving as food to a human or another animal, e.g., a cow, a calf, a steer, a chicken, a turkey, a goose, a duck, a sheep, or a pig.

For a mammal, such as, e.g., a human, an intranasal dosage or dosage for oral inhalation of the electrolyzed saline solution may vary from between about 0.01 ml/kg/day body weight to about 10 ml/kg/day body weight. In one embodiment, the inhaled or intranasal dosage of the electrolyzed saline solution is between about 0.25 to about 4 ml/kg/day body weight, such as, e.g., from about 0.5 to 3.0 ml/kg/day, such as, e.g., from about 0.25, about 0.3, about 0.4, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, or about 4.0 ml/kg/day body weight.

For a mammal, such as, e.g., a human, an intravenous injection dosage of the electrolyzed saline solution may vary from between about 0.01 ml/kg/day body weight to about 10 ml/kg/day body weight. In one embodiment, the i.v. injection dosage of the electrolyzed saline solution is between about 0.25 to about 4 ml/kg/day body weight, such as, e.g., from about 0.5 to 3.0 ml/kg/day, such as, e.g., from about 0.25, about 0.3, about 0.4, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, or about 4.0 ml/kg/day body weight.

The doses can be divided into smaller doses and administered two or more times per day or may be administered in a single dose. The regimen can vary according to the indication being treated. For example, it may be advantageous to administer the electrolyzed saline solution for several days followed by a rest period and repeating the cycle for as long as necessary or as indicated by test results. A typical regimen might be five days of treatment followed by two days rest with the cycle repeated for two months. Depending on clinical status or laboratory tests, this regimen may be reduced to, e.g. three days of treatment per week for six weeks. These regimens are exemplary only and are not meant to be limiting as any number or variation might be dictated according to circumstances.

In certain situations where it may be desired to utilize higher concentrations of chlorine and oxygen active agents produced from the electrolysis of a saline solution, it may be desirable to concurrently administer in vivo or subject a solution in vitro to modulating or moderating chemicals. These modulating chemicals are administered before, concurrent with or after the electrolyzed saline and may be administered in any suitable manner, such as, e.g., intravenously, parenterally, intranasally, or orally. As used herein the terms “moderating”, “modulating” and “neutralizing” agents may be used interchangeably.

The modulating chemicals are enzymes or reducing agents that interact with and reduce the active microbicidal agents to innocuous compounds. The enzymes are inclusive of, but not limited to, the superoxide dismutases (SOD), catalase and glutathione peroxidase. These oxygen radicals are converted to hydrogen peroxide by Cu/Zn activated superoxide dismutases (SOD) in the cells. In a properly functioning system the hydrogen peroxide is then converted to oxygen and water by a catalase. If the hydrogen peroxide and the superoxide radical are allowed to combine, the more deadly hydroxide radical is formed.

Administration

The electrolyzed saline solution of the present invention can be suitable for the treatment of bacterial, viral and fugal related syndromes and immunological disorders. Examples of such syndromes and/or immunological disorders for which either in vitro or in vivo treatment could be beneficial are Epstein-Barr virus, hepatitis A, B and C, rhinovirus, rubeola, rubella, parvovirus, papilloma virus, influenza and parainfluenza viruses, enteroviruses; Herpes simplex viruses; Varicella-zoster viruses, Adenoviruses, respiratory syncytial viruses, alphaviruses, flaviviruses, retroviruses (including AIDS and AIDS related syndromes), bacteremia, septicemia, fungal infections, parasitic infections (nematodes, trematodes, protozoal, e.g., Cryptosporidium helminthic), mycobacterial infections, bacterial Gram positive and Gram negative superficial and systemic infections and other viral, bacterial and/or fungal associated diseases.

There are also situations where fluids can be beneficially treated in vitro, to purify, decontaminate, or otherwise render such fluid acceptable for administration to a warm-blooded host. For example, the blood supply taken from donors at blood banks has been found on occasion to be contaminated by the HIV virus and other organisms such as hepatitis A, B and C viruses, CMV (cytomegalovirus), and bacteria (such as Yersinia). Any treatment of whole blood, plasma or cell isolates to render them benign from infectious organisms without destroying the therapeutic characteristics of such fluids would be very beneficial.

The electrolyzed saline solution is suitable for the treatment or prevention of respiratory disorders and airway inflammation, including cystic fibrosis and asthma and asthma-related inflammation, as described herein. A “respiratory disorder,” as used herein, can be any disorder or abnormality involving the respiratory system, including disorders or abnormalities that are linked with (i.e., cause, caused by, or associated with) airway inflammation and/or mucus over-production in the respiratory tract. For example, the term “respiratory disorder” includes disorders such as, e.g., asthma and cystic fibrosis.

In the context of the present invention, the word “treatment” can mean any positive change in the symptoms or pathology of the treated disorder, such as, e.g., the complete eradication of the disorder, a discontinuance in the negative progression of the disorder, a reduction in the severity of symptoms, increase in the patient's quality of life, and/or extension of the patient's life.

The electrolyzed saline solution can comprise any amount of ozone and active species suitable for treating or preventing respiratory disorders and airway inflammation, including cystic fibrosis, asthma and asthma-related inflammation, in an animal, such as, e.g., about 0.1 ppm to about 100 ppm ozone and about 5 ppm to about 300 ppm of at least one active chlorine species. The solution can also comprise about 0.1 ppm to about 300 ppm of at least one active oxygen species, and/or about 5 ppm to about 300 ppm of at least one active hydrogen species. In one embodiment, the solution comprises about 0.1 ppm to about 30 ppm ozone and about 10 ppm to about 100 ppm of at least one active chlorine species, and can also comprise about 0.1 ppm to about 100 ppm of at least one active oxygen species, and/or about 10 ppm to about 100 ppm of at least one active hydrogen species. In one embodiment, the solution comprises about 9 ppm to about 15 ppm ozone and about 10 ppm to about 80 ppm of at least one active chlorine species, and can also comprise about 0.1 ppm to about 80 ppm of at least one active oxygen species, and/or about 10 ppm to about 80 ppm of at least one active hydrogen species. In one embodiment, the electrolyzed saline solution comprises less than about 0.8 ppm ozone and about 10 ppm to about 80 ppm of at least one active chlorine species, such as, e.g., about 55 ppm to about 80 ppm of at least one active chlorine species. In yet another embodiment, the solution comprises about 0.30 to about 0.7 ppm ozone and about 10 ppm to about 80 ppm of at least one active chlorine species.

In one embodiment, the active chlorine species comprises at least one of an active chlorine species selected from the group consisting of: free chlorine, hypochlorous acid and hypochlorite ion. In one embodiment, the active oxygen species is O₂³. In one embodiment, the active hydrogen species is H₂O₂. In one embodiment, the solution is prepared by subjecting a 1% or less saline solution, such as, e.g., 0.9% NaCl (w/vol), 0.45% NaCl (w/vol), and 0.215% NaCl (wt/vol), to electrolysis under conditions sufficient to produce the desired active ingredients.

The electrolyzed saline solution can be administered in any suitable manner. In one embodiment, the method for treating or preventing respiratory disorders and airway inflammation, including cystic fibrosis, asthma and asthma-related inflammation, in an animal comprises administering the electrolyzed saline solution intranasally or via oral inhalation to an animal, such as, e.g., a human or a food animal. The food animal can be any animal suitable for serving as food to a human or another animal, such as, e.g., a cow, a calf, a steer, a chicken, a turkey, a goose, a duck, a sheep, or a pig. In one embodiment, the method for treating or preventing respiratory disorders and airway inflammation, including cystic fibrosis, asthma and asthma-related inflammation, in an animal comprises administering the electrolyzed saline solution in the form of an inhaler to an animal, such as, e.g., a human or a food animal. In one embodiment, the electrolyzed saline solution can be administered by intravenous or intraperitoneal injection in a suitable amount, such as, e.g., amounts described herein.

In one embodiment, the administration of the electrolyzed saline solution to a food animal minimizes the risk of antimicrobial-resistant pathogens developing in the food chain. In one embodiment, the administration of the electrolyzed saline solution to a human minimizes the risk of the development of antimicrobial-resistant pathogens. Administration of the electrolyzed saline solution exhibits no apparent toxicity or tissue residue.

Animal Model

An animal model of airway disease having a pathology similar to that of cystic fibrosis, such as, e.g., over-secretion of mucus, is also provided. In one embodiment, the animal model further comprises Pseudomonas aeruginosa infection.

Cystic fibrosis is characterized by excess mucus production, especially in the lungs, pancreas and intestines. Furthermore, cystic fibrosis patients are prone to lung infections, especially from Pseudomonas aeruginosa, which is associated with progressive deterioration of lung function in the patients. As such, an animal model exhibiting mucus over-production and/or airway inflammation that further comprises Pseudomonas aeruginosa infection is especially useful in elucidating new treatments for patients with cystic fibrosis. In one embodiment, the animal model can be used to determine the effectiveness of a treatment, such as, for example, the administration of electrolyzed saline solution, on symptoms associated with mucus over-production and/or Pseudomonas aeruginosa infection in the animal model.

In one embodiment, the respiratory system of the animal model has many of the histologic characteristics of the respiratory system of human cystic fibrosis patients. Such histologic characteristics include characteristics suitable to demonstrate the cystic fibrosis-like qualities of the animal model, such as, for example, bacteria Pseudomonas aeruginosa infection, mucus secretion, lung edema, lung hemorrhage, and lung infiltration by polymorphonuclear leukocytes (PMNs) and eosinophils.

In one embodiment, a murine model having a cystic fibrosis-like syndrome is disclosed. In one embodiment, the cystic fibrosis-like mouse model is developed using ovalbumin (OVA)-induced chronic asthmatic mice, which are then infected with Pseudomonas aeruginosa such that a lung disease state comparable with that of a cystic fibrosis patient is established.

Methods for inducing a cystic fibrosis-like syndrome in a mouse are also provided. In one embodiment, the present invention provides a method for inducing a cystic fibrosis-like syndrome in a mouse comprising: a) inducing chronic asthma in a mouse and b) administering bacteria to the mouse of step a) such that the mouse develops a cystic fibrosis-like syndrome. In one embodiment, the chronic asthma is induced by a suitable allergen immunization/challenge protocol, such as, e.g., a protocol involving administration of ovalbumin to the mouse. Suitable allergen immunization/challenge protocols are described herein.

The bacteria can be any bacteria suitable for infecting the animal, such as, e.g., Staphylococcus (e.g., S. aureus); Streptococcus (e.g., Group A, Group B, Group C, or Group D, such as, e.g., S. pyogenes, S. agalactiae, S. milleri, S. pneumoniae); Enterococcus; Corynebacterium; Bacillu; Listeria; Clostridium; Mycobacterium; Actinomyces; Enterobacteriaceae (e.g., E. Coli); Proteus; Klebsiella; Serratia; Enterobacter; Salmonella; Shigella; Pseudomonas (e.g., P. aeruginosa); or any other suitable bacteria.

In one embodiment, the bacteria is Pseudomonas aeruginosa. In one embodiment, the Pseudomonas aeruginosa is a strain isolated from a human cystic fibrosis patient, such as, for example, Pseudomonas aeruginosa strains CF 18. In one embodiment, the cystic fibrosis-like syndrome comprises one or more characteristics of human cystic fibrosis, such as, e.g., bacterial infection, mucus secretion, lung edema, lung hemorrhage, and lung infiltration by polymorphonuclear leukocytes (PMNs) and eosinophils.

In one embodiment, the present invention provides a method for inducing a cystic fibrosis-like syndrome in a mouse comprising: a) administering ovalbumin to a mouse such that a chronic asthma condition develops in the mouse and b) administering Pseudomonas aeruginosa to the mouse of step a) such that the mouse develops a cystic fibrosis-like syndrome. In one embodiment, the cystic fibrosis-like syndrome comprises one or more characteristics of human cystic fibrosis, such as, e.g., bacterial infection, mucus secretion, lung edema, lung hemorrhage, and lung infiltration by polymorphonuclear leukocytes (PMNs) and eosinophils. In one embodiment, the Pseudomonas aeruginosa is a strain isolated from a human cystic fibrosis patient, such as, e.g., strains described herein.

The ovalbumin can be administered in any suitable manner and in any suitable amount to induce chronic asthma in the mouse. In one embodiment, ovalbumin is administered on one or more of days 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, and/or 31 in a month. The ovalbumin can be administered in any suitable manner, such as, e.g., intraperitoneally, intranasally, or in a combination of manners. In one embodiment, the ovalbumin is administered intraperitoneally on days from about 0 to about 14 and is administered intranasally on days from about 14 to about 31, such that chronic asthma is induced in the animal. The ovalbumin can be administered in any suitable amount to induce chronic asthma in the animal, such as e.g., about 15 mg, about 20 mg, about 25 mg, about 30 mg, about 40 mg, about 50 mg, about 60 mg, about 70 mg, about 80 mg, about 90 mg, about 100 mg, about 125 mg, about 150 mg, about 175 mg, about 180 mg, or more per dose. In one embodiment, the ovalbumin is complexed with alum before administration.

The Pseudomonas aeruginosa can be administered in any suitable manner and in any suitable amount to induce bacterial infection in the mouse. The Pseudomonas aeruginosa can be administered in any suitable manner to induce infection in the mouse, such as, e.g., intranasally, intraperitoneally, or in a combination of manners. The Pseudomonas aeruginosa can be administered in any suitable amount to induce infection in the animal, such as e.g., about 1×10³, about 1×10⁴, about 1×10⁵, about 1×10⁶, about 1×10⁷, about 1×10⁸, or more per dose.

Methods of screening pharmaceutical agents for effectiveness in the treatment of cystic fibrosis using animal models disclosed herein are also provided. In one embodiment, the method comprises a) inducing chronic asthma in a mouse; b) administering bacteria to the mouse of step a) such that the mouse develops a cystic fibrosis-like syndrome; c) treating the mouse with the pharmaceutical agent of interest; and d) evaluating the characteristics of the cystic fibrosis-like syndrome to determine if there is a difference in the characteristics of the syndrome after treatment with the pharmaceutical compared with the characteristics of the syndrome prior to treatment with the pharmaceutical; wherein the effectiveness of the pharmaceutical agent in the treatment of cystic fibrosis is demonstrated by a positive change in the characteristics of the syndrome after treatment with the pharmaceutical compared to the characteristics of the syndrome prior to treatment. In one embodiment, a positive change in the characteristics or pathology of the syndrome comprises an improvement in the disorder, such as, e.g., the complete eradication of the disorder, a discontinuance in the negative progression of the disorder, a reduction in the severity of symptoms, increase in quality of life, and/or extension of life. In one embodiment, the chronic asthma is induced by a suitable allergen immunization/challenge protocol, such as, e.g., a protocol involving administration of ovalbumin to the mouse. In one embodiment, the bacteria is Pseudomonas aeruginosa. In one embodiment, the Pseudomonas aeruginosa is a strain isolated from a human cystic fibrosis patient, such as strains described herein. In one embodiment, the cystic fibrosis-like syndrome comprises one or more characteristics of human cystic fibrosis, such as, e.g., bacterial infection, mucus secretion, lung edema, lung hemorrhage, and lung infiltration by polymorphonuclear leukocytes (PMNs) and eosinophils.

The invention is further illustrated by the following examples, which of course should not be construed as in any way limiting its scope.

### EXAMPLES

### Example 1

This example demonstrates the anti-inflammatory effects and clearance of hypersecreted mucus in a murine asthma model upon administration of an effective amount of electrolyzed saline solution.

Reagents

Crystalline OVA was obtained from Pierce Chemical Co. (Rockford, Ill.), aluminum potassium sulfate (alum) from Sigma Chemical Co. (St. Louis, Mo.), pyrogen-free distilled water from Baxter Healthcare Corporation (Deerfield, Ill.), and 0.9% sodium chloride (normal saline) from Lyphomed (Deerfield, Ill.). The OVA (500 μg/ml in normal saline) was mixed with equal volumes of 10% (wt/vol) alum and distilled water. The mixture was brought to pH 6.5 using 10 N NaOH. After incubation for 60 minutes at room temperature, the mixture was centrifuged at 750 g for 5 minutes. The resulting pellet was resuspended to the original volume in distilled water and used within 1 hour.

Allergen Immunization/Challenge Protocol

Mice (BALB/c; Jackson Laboratory, Bar Harbor, Me.) received an i.p. injection of 0.2 ml (100 mg) of OVA complexed with alum on day 0 and 14. On days 14, 25, 26, and 27, mice were anesthetized with 0.2 ml i.p. of ketamine (0.44 mg/ml)/xylanzine (6.3 mg/ml) in normal saline before receiving an intranasal (i.n.) dose of 100 mg OVA in 0.05 ml normal saline on days 25, 26, and 27. Two control groups were used. The first group received normal saline with alum i.p. on days 0 and 14 and normal saline without alum i.n. on days 14, 25, 26, and 27. The second group received OVA with alum i.p. on days 0 and 14, OVA without alum i.n. on day 14 and normal saline alone on days 25, 26, and 27.

BALB/c mice given i.p. OVA in alum twice over a 14 d period followed by three i.n. doses of OVA on day 25, 26, and 27 of the protocol developed OVA-specific IgE and histologic and physiologic founding mimicking human asthma. In mice examined 24 hours after the last i.n. administration of OVA on day 28, eosinophils were the predominant inflammatory cells in both the lung interstitium and BAL fluid. Mucus occlusion of the airway lumen in the OVA-treated mice was a prominent feature of this allergen induced inflammatory disease. In this murine model, a marked increase in airway mucus is observed on day 22 by day 24 to 25, airway epithelial cells are extensively replaced by mucus-producing goblet cells (Budhecha, S., et al., Am. J. Respir. Crit. Care Med., 155: A754 (1997)).

Drug Treatment

Electrolyzed saline solution at 3 different concentrations was produced the day before use. 0.9% sodium chloride, USP (Baxter Healthcare Co., Deerfield, Ill.) was used and the electrolyzed solution pH was adjusted to pH 7.4 using 2N HCl. Chlorine and hydrogen peroxide were tested and recorded. In the experiments, a 50 ml dose of electrolyzed saline solution was administered intranasally to OVA-treated mice before each challenge. After 30 min, the mice were then challenged with OVA at day 25, 26, and 27.

TABLE 1

| | Treatment | | | | | |
|---|---|---|---|---|---|---|
| | MDI-P 100 | MDI-P 50 | MDI-P 25 | | | |
| | with OVA | with OVA | with OVA | | OVA | MDI-P |
| | challenge | challenge | challenge | Saline | only | only |
| # of | 6 | 6 | 6 | 6 | 6 | 6 |
| Animals | | | | | | |

Bronchoalveolar Lavage

After tying off the left lung at the mainstem bronchus, the right lung was lavaged three times with 0.4 ml of normal saline. Bronchoalveolar lavage (BAL) fluid cells from a 0.05 ml aliquot of the pooled sample were counted using a hemocytometer and the remaining fluid was centrifuged at 4° C. for 10 min at 200 g. The supernatant was stored at −70° C. until eicosanoid analyses were performed. After resuspension of the cell pellet in normal saline containing 10% BSA, BAL cell smears were made on glass slides. To stain eosinophils, dried slides were stained with Discombe's diluting fluids (0.05% aqueous eosin and 5% (vol/vol) acetone in distilled water) for 5-8 minutes, rinsed with water for 0.5 minutes and counterstained with 0.07% methylene blue for 2 minutes.

Lung Histology

After BAL, the trachea and left lung (upper and lower lobes) were obtained and fixed in Carnoy's solution at 20° C. for ˜15 hours. After embedding in paraffin, the tissues were cut into 5 mm sections and stained with Discombe's solution and counter-stained with methylene blue as described above. The eosinophil number per unit airway area (2,200 mm²) was determined by morphometry as previously described. Airway mucus was identified by the following staining methods: methylene blue, hematoxylin and eosin, mucicarmine, toluidine blue, alcian blue, and alcian blue/periodic acid-Schiff (PAS) reaction. Henderson, W. R., et al., J. Exp. Med., 184: 1483-1494 (1996). Mucin was stained with mucicarmine solution and metanil yellow counterstain was employed. Mucin and sialic acid-rich nonsulfated mucosubstances were stained metachromatically with toluidine blue, pH 4.5. Acidic mucin and sulfated mucosubstances were stained with alcian blue, pH 2.5; nuclear fast red counterstain was used. Neutral and acidic mucosubstances were identified by alcian blue, pH 2.5, and PAS reaction. The degree of mucus plugging of the airways (0.5-0.8 mm in diameter) was also assessed by morphometry. The percent occlusion of airway diameter by mucus was classified on a semi-quantitative scale from 0 to +++++ as described in the Figure Legends. The histologic and morphometric analyses were performed by individuals blinded to the protocol design.

Assay of Airway Mucus Glycoproteins

Mucus glycoproteins in BAL fluid were assayed by slot plotting and PAS staining. Airway mucus release in both lower and upper pulmonary airways was identified by special stains. Nitrocellulose membranes (0.2 mm pore size; Schleicher & Schuell, Keene, N.H.) were wetted in distilled water and then in normal saline before placement in a Minifold II 72-well slot blot apparatus. The PAS staining was done and the images were captured and digitized by a ScanJet Ilcx Scanner with HP DeskScan II software (Microsoft, Windows Version) (Hewlett Packard, Palo Alto, Calif.). This system was linked to a Dell Dimension XPS P90 computer (Dell Corporation, Austin, Tex.) employing Image Pro Plus, Version 1.1 for Windows software (Media Cybernetics, Silver Spring, Md.). The images were assessed on a 256 gray level scale using a Dell UltraScan 17ES monitor with extra high-resolution graphic mode (1,280×1,024 pixels, 78.9 kHz horizontal scanning frequency, 74 Hz vertical scanning frequency). The integrated intensity of the PAS reactivity of the BAL samples was quantitated by comparison to the standard curve for human respiratory mucin as previously described.

Effect of Electrolyzed Saline Recruitment into BAL Fluid

Twenty-four hours after the final intranasal OVA challenge, BAL was performed on the right lung of all animals from each experimental group. The left lung tissue was obtained to assess inflammatory cell infiltration and mucus release. The effect of electrolyzed saline on the airway inflammation was determined.

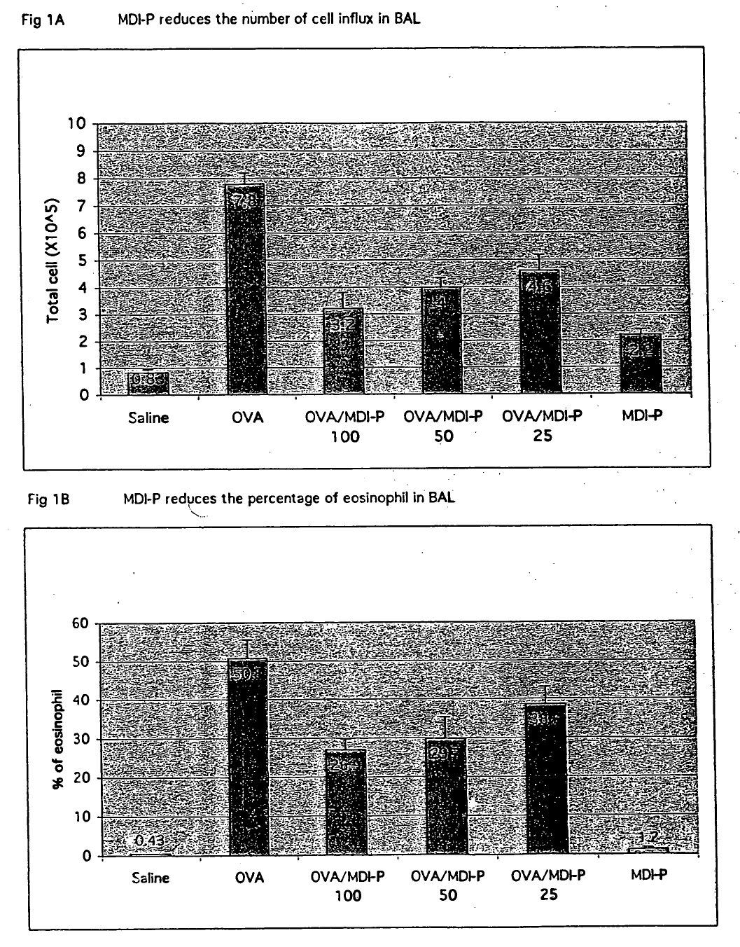

OVA-sensitized/challenged mice had a 9.3 fold increase in total BAL fluid cells compared with saline group (FIG. 1A). A total of 50% of the BAL fluid cells were eosinophils in the OVA-treated mice compared with less than 1% of the total BAL fluid cells in the saline-treated control. The electrolyzed saline solution alone control has about 1.2% of eosinophils (FIG. 1B).

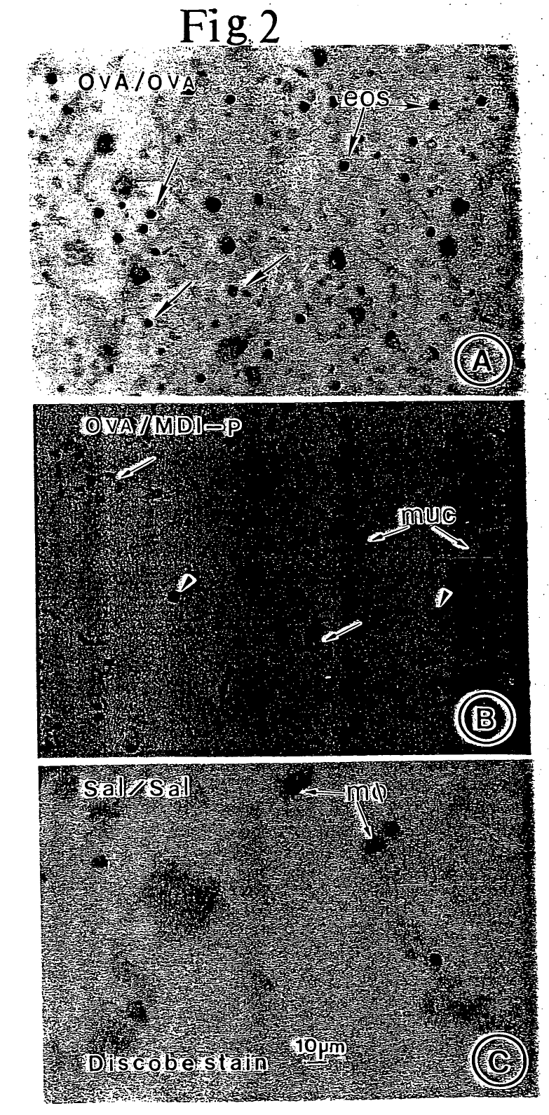

As shown in FIGS. 2A, 2B and 2C, in OVA-sensitized/challenged mice, treatments with electrolyzed saline solution at 3 different concentrations reduced the influx of eosinophils into the BAL fluid by 30-45% (FIG. 2A, 2B, 2C). FIG. 2A is a photograph showing BAL lavage fluids from an OVA-treated mouse challenged at days 25, 26, and 27. The photograph shows that the lung-lavaged fluid contains more than 50% eosinophils (EOS) and by staining in the modified compi-stain (arrows). FIG. 2B is a photograph showing BAL lavage fluids from a mouse treated with OVA and challenged and dosed with MDI-P 100. The photograph shows that there are fewer eosinophils (arrowheads) and that many of the mucus aggregates are absorbed in the fluid. FIG. 2C is a photograph showing BAL lavage fluids from the lungs of a mouse treated with saline only. The photograph shows that most of the cells are macrophages.

Inflammatory Cell and Eosinophil Infiltration of Lung Interstitium

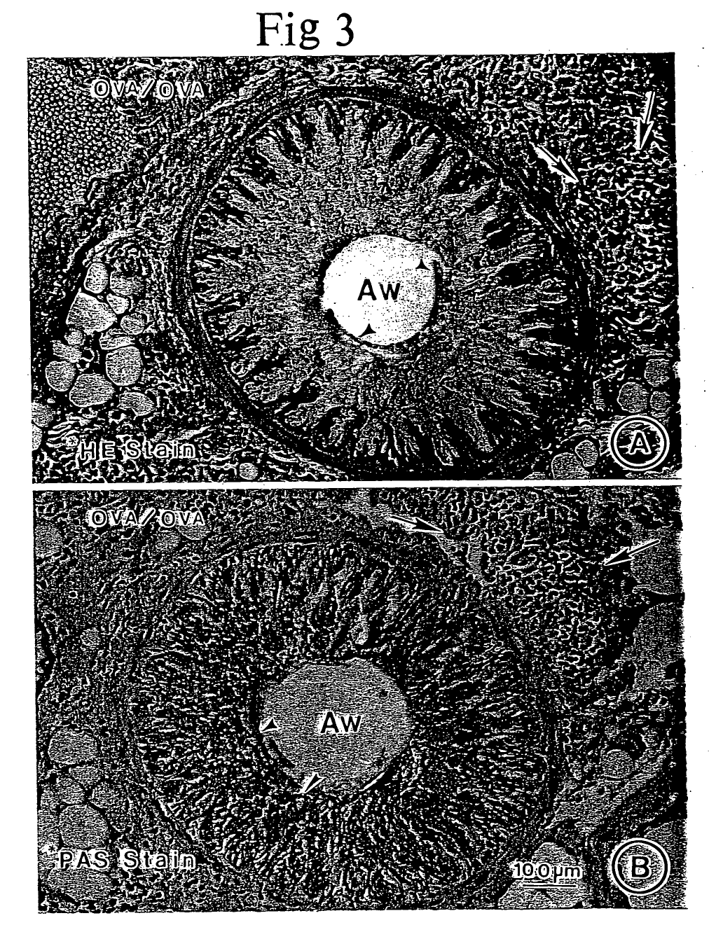

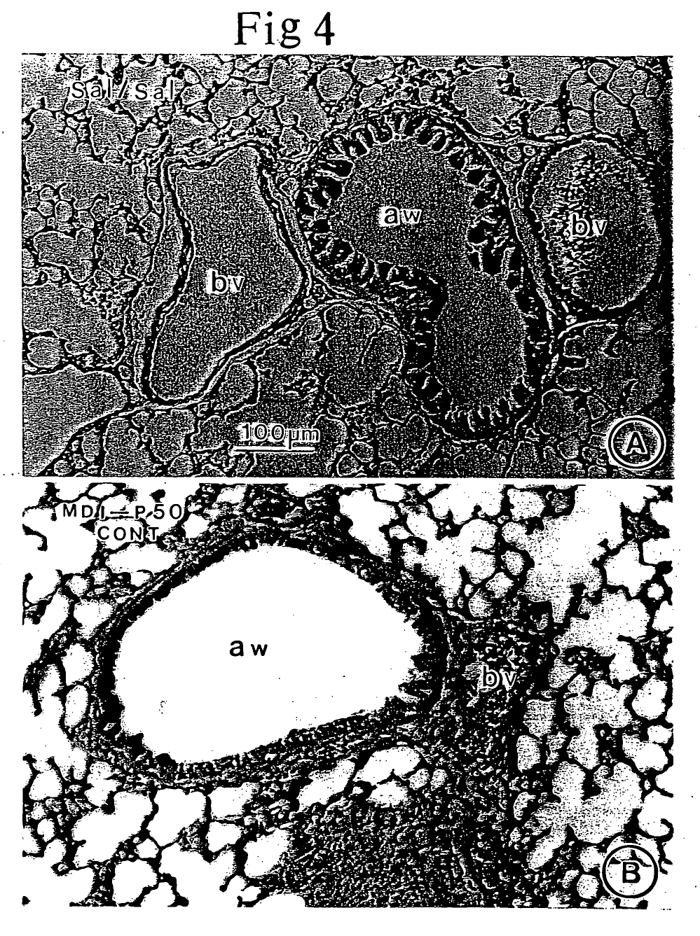

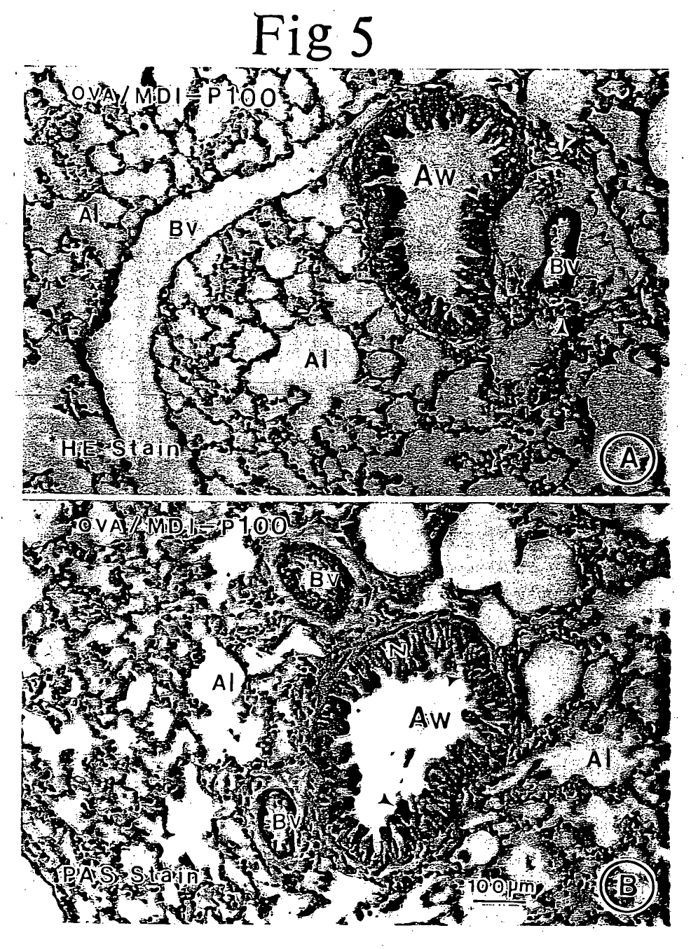

Inflammatory cell and eosinophil infiltration of lung interstitium is less in electrolyzed saline solution treated mice than in mice who received OVA treatment alone. A marked infiltration of eosinophil and mononucleous cells around the airways and pulmonary vessels was observed in the lung interstitium of OVA treated mice compared with electrolyzed saline solution alone or saline controls as determined by light microscopy (FIG. 3AB, FIG. 4AB, FIG. 5AB).

FIG. 3A is a photograph of H&E stained lung tissue from an OVA-immunized mouse that was challenged 3 times at days 25, 26, and 27. The photograph shows the lung tissue changes due to the pulmonary allergic reaction to the airway. The photograph shows the occlusion of the airway (AW) and that the airway is plugged with amorphous materials (arrowheads). The photograph also shows that there are many cells that have infiltrated the airway interstitium (arrows). FIG. 3B is a photograph of a PAS-stained lung tissue section tissue from an OVA-immunized mouse that was challenged 3 times at days 25, 26, and 27 showing the airway (AW) is filled with carbohydrate-mucosubstances (arrowheads) by the staining pattern of the red Shiff's reagent.

FIG. 4A is a photograph of lung tissue from a mouse treated with saline only showing that the airway (aw) is very clear and that there are no inflammatory cells in or around the airway and blood vessels (BV). FIG. 4B is a photograph of lung tissue from a mouse treated with MDI-P 50 without OVA immunization showing the lung tissue appears normal. The photograph also shows that the airway (aw) is very clear of mucus and no inflammatory cells are seen in the interstitium of the airway or blood vessels (BV).

FIG. 5A is a photograph of lung tissue from mice that were OVA-sensitized/challenged and treated with MDI-P 100. The photograph shows that the airway (AW) is very clear with a slightly constricted appearance, and that a small number of inflammatory cells (arrowheads) appeared around the blood vessel (BV). The alveoli (AL) also appeared normal. FIG. 5B is a photograph of lung tissue from mice that were OVA-sensitized/challenged and treated with MDI-P 100. The photograph shows the airway (AW) has a fewer number of mucus cells on the lumenal surface (arrowheads). Blood vessels (BV) and alveoli (AL) did not appear to be inflamed.

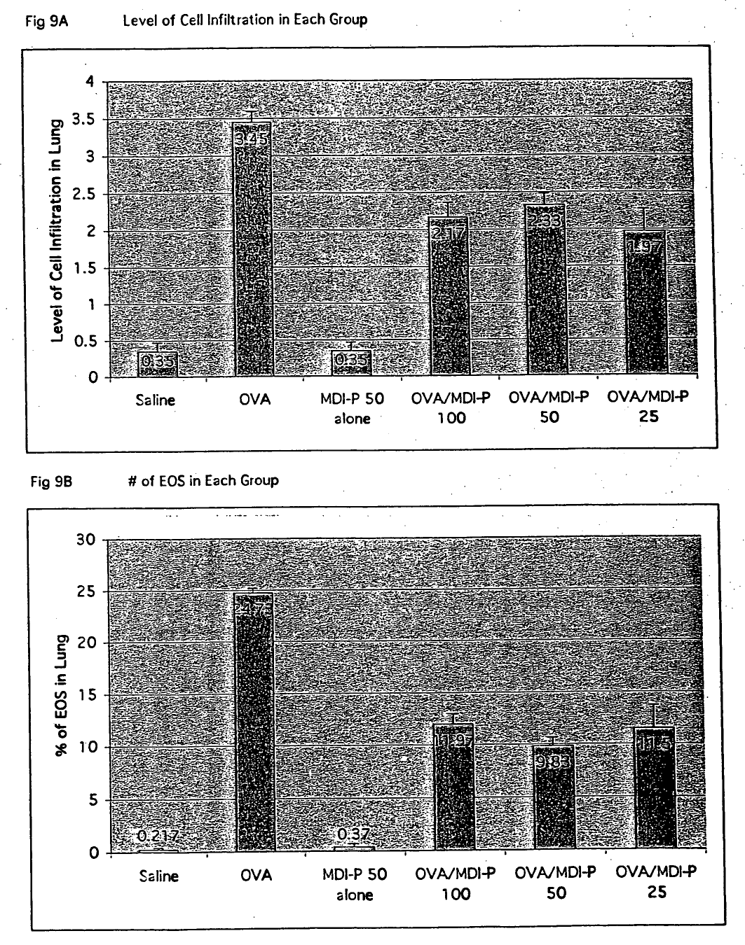

The eosinophil influx with the interstitium was reduced 50% by airway mucus hypersecretion with electrolyzed saline solution treatment (FIG. 9), as determined by morphometric analysis.

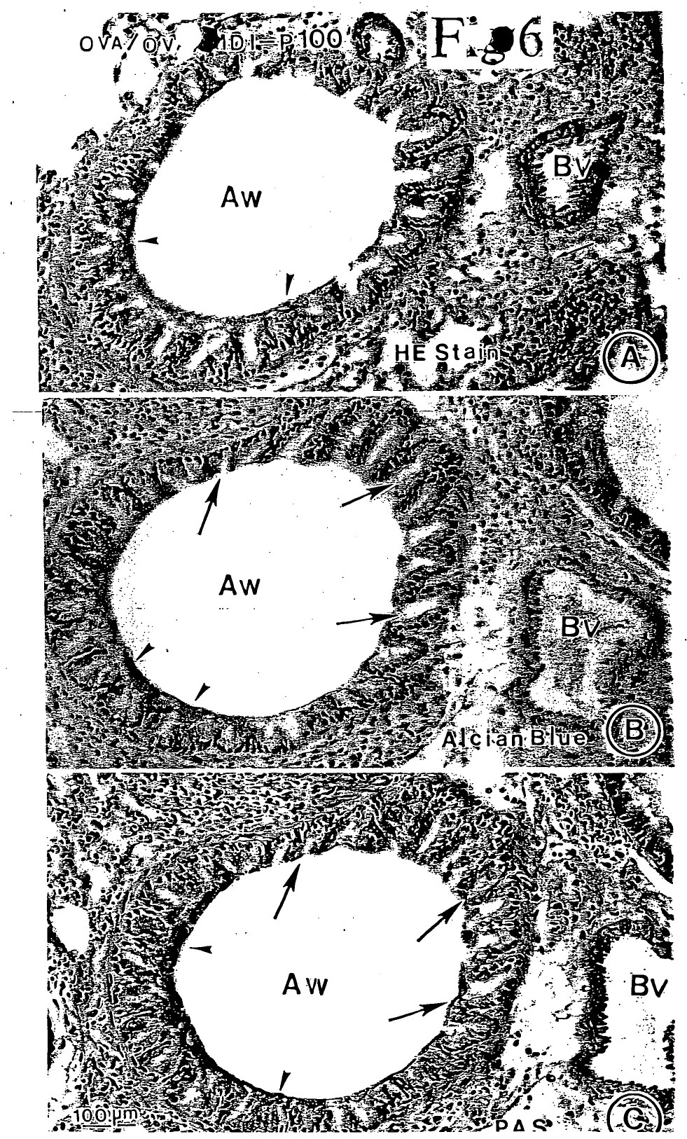

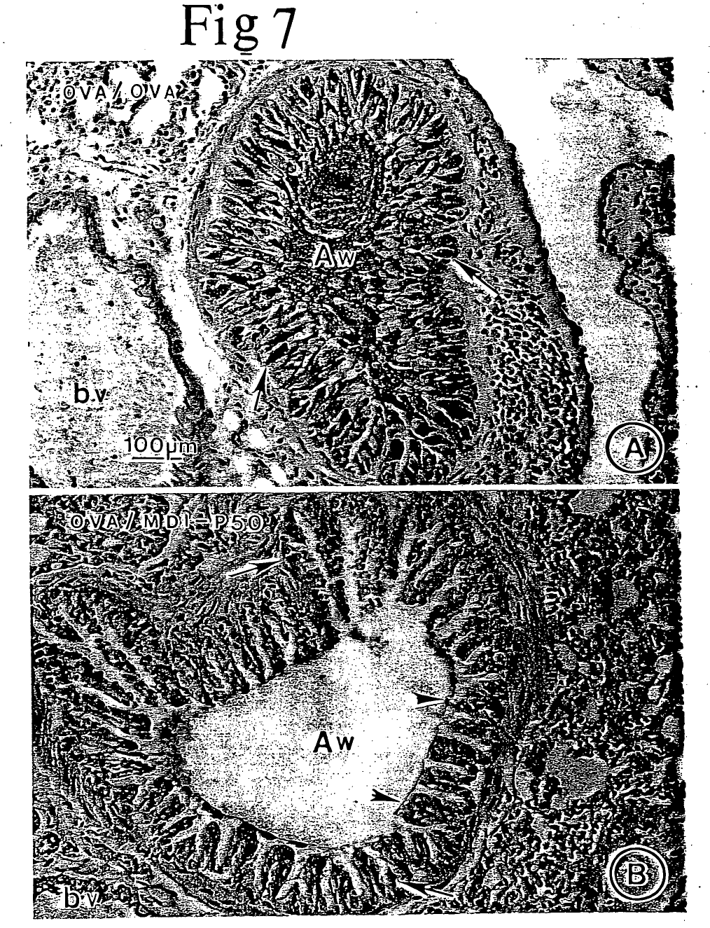

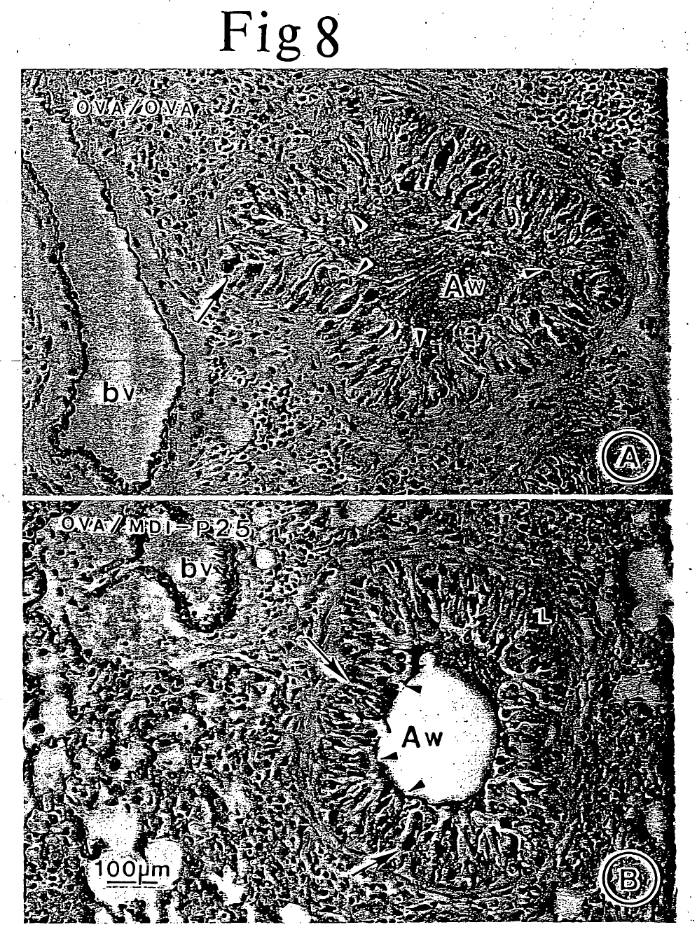

Hyperplasia of airway mucus cells and hypersecretion of mucus were seen in the OVA-treated mice (FIG. 3A, 3B vs. FIG. 5). Mucus gland hyperplasia is less with electrolyzed saline solution treatment (FIG. 3 vs. FIG. 6) and occlusion of airway was reduced by treatment with electrolyzed saline 100% solution (FIG. 6), electrolyzed saline 50% solution (FIG. 7) and electrolyzed saline 25% solution (FIG. 8).

FIG. 6A is a photograph demonstrating histologic evidence of airway inflammation reduction in the lungs of mice treated with MDI-P 100. The photograph shows that less mucus release in the airway (AW) is evident (arrowheads) and that eosinophils are observed less frequently in the interstitium (arrows). FIG. 6B is a photograph demonstrating histologic evidence of airway inflammation reduction in the lungs of mice treated with MDI-P 100. The photograph shows alcian blue stains the mucus in the airway (arrowheads) of mice treated with MDI-P 100% with less frequency that the lung of mice treated with OVA only and that a portion of the airway surface is clear of mucus (arrows). FIG. 6C is a photograph demonstrating histologic evidence of airway inflammation reduction in the lungs of mice treated with MDI-P 100. The photograph shows PAS staining of the glycoproteins (arrowheads) and fewer mucus cells in the airway (AW) epithelial layer (arrows).

FIG. 7A is a photograph showing lung tissue from OVA-treated mice showing the airway (AW) is plugged by mucus and that hyperplasia mucus cells (arrows) are the predominant feature in the OVA-immunized/challenged airways. FIG. 7B is a photograph showing lung tissue from MDI-P 50 and OVA immunized and challenged mice showing less mucus secretion (arrowheads) and mucus cell hyperplasia (arrows).

FIG. 8A is a photograph showing lung tissue from OVA sensitized/challenged mice demonstrating airway (AW) mucus plugging (arrowheads) and that an intensive mucus cell hyperplasia of the airway (arrows) is evident. FIG. 8B is a photograph showing lung tissue from OVA-sensitized/challenged mice treated with MDI-P 25. The photograph demonstrates the reduction of airway mucus (arrowheads) and mucus cell hyperplasia (arrows). Airway lumen occlusion by mucus was greater in smaller diameter airways. These changes were absent in the saline-treated (FIG. 4B) control animals.

Mucus Accumulation Reduced by MDI-P

Cross-sections of the upper and lower lobes of left lung of OVA-treated and control mice were examined by light microscopy for mucus accumulation to compare with electrolyzed saline solution treated mice. By morphometry analyses, more that 70% of the airways of control animals treated either with saline or electrolyzed saline solution alone had no evidence of airway mucus release (FIG. 4, 5). In contrast, OVA-immunized/challenged mice had morphologic evidence for widespread mucus occlusion of the airways (FIG. 3, FIG. 7A, FIG. 8A). Electrolyzed saline solution treatments reduced the airway mucus release in the OVA-treated mice (FIG. 6, FIG. 7B, and FIG. 8B).

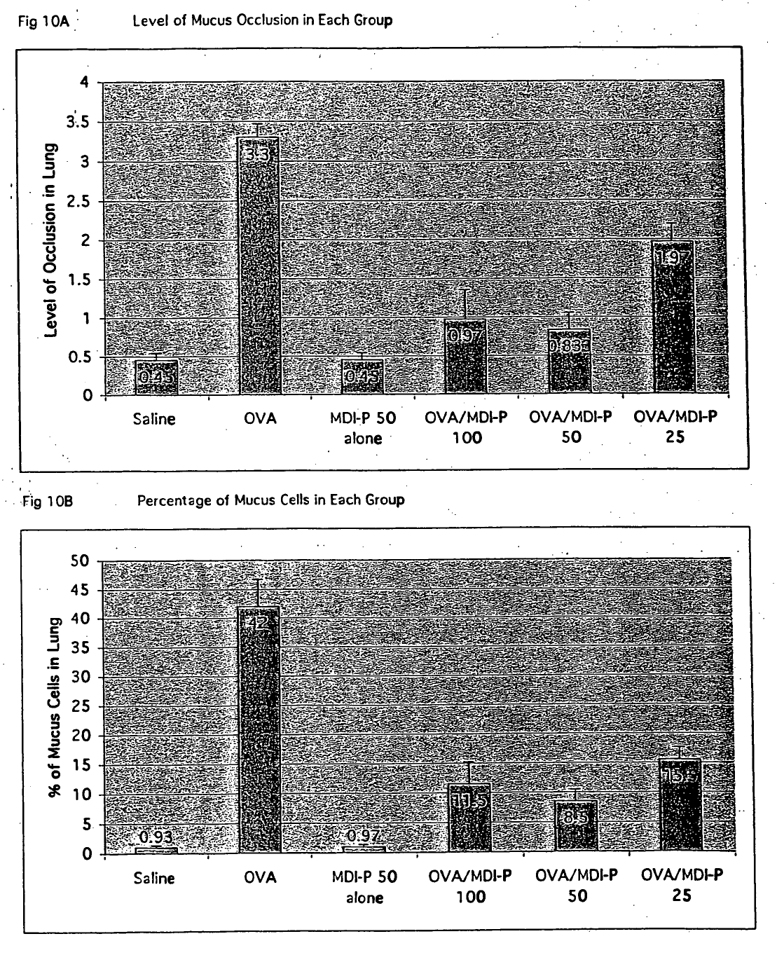

This example demonstrates that electrolyzed saline solutions are useful and beneficial agents for reducing allergen-induced airway eosinophil infiltration and mucus release in a mouse model of asthma. In this example, electrolyzed saline solution administration was found to have a very beneficial effect on the airway inflammation. Administration of electrolyzed saline solution reduces the influx of eosinophils into the lung and the BAL, which is a key feature of asthma. Furthermore, this example shows that MDI-P blocks mucus cell hyperplasia and reduces the airway mucus hypersecretion (FIG. 10).

### Example 2

This example demonstrates the effect of administration of an electrolyzed saline solution in a mouse model of CF-like lung infection and inflammation.

Reagents

Crystalline OVA was obtained from Pierce Chemical Co. (Rockford, Ill.), aluminum potassium sulfate (alum) from Sigma Chemical Co. (St. Louis, Mo.), pyrogen-free distilled water from Baxter Healthcare Corporation (Deerfield, Ill.), and 0.9% sodium chloride (normal saline) from Lyphomed (Deerfield, Ill.). The OVA (500 μg/ml in normal saline) was mixed with equal volumes of 10% (wt/vol) alum in distilled water. The mixture was brought to pH 6.5 using 10 N NaOH. After incubation for 60 minutes at room temperature, the mixture was centrifuged at 750 g for 5 minutes. The resulting pellet was resuspended to the original volume in distilled water and used within 1 hour.

Animals

All animal use procedures were approved by the Animal Care Committee. Female BALB/c mice were obtained. (6-8 weeks of age at purchase; The Jackson Laboratory, Bar Harbor, Me.).

Allergen Immunization/Challenge Protocol

Mice received an i.p. injection of 0.2 ml (100 mg) of OVA complexed with alum on day 0 and 14. On days 14, 25, 26, and 27, mice were anesthetized with 0.2 ml i.p. of ketamine (0.44 mg/ml)/xylanzine (6.3 mg/ml) in normal saline before receiving an intranasal (i.n.) dose of 100 mg OVA in 0.05 ml normal saline on days 25, 26, and 27. Two control groups were used. The first group received normal saline with alum i.p. on days 0 and 14 and normal saline without alum i.n. on days 14, 25, 26, and 27. The second group received OVA with alum i.p. on days 0 and 14, OVA without alum i.n. on day 14 and normal saline alone on days 25, 26, and 27.

Bacteria and Growth Conditions

Initial studies employed P. aeruginosa strain CF 18 (provided by Dr. S. Lory, University of Washington), which is a non-mucoid variant isolated from a 2 year old patient with cystic fibrosis. Bacteria were grown statically in Luria broth (1% tryptone, 0.5% yeast extract, 0.5% sodium chloride) and supplemented with 5 mM magnesium chloride, at 37° for 16 hours. Prior to their aerosol or i.n. delivery to mice, the bacteria were sedimented by centrifugation at 5000×g for 5 minutes, washed once with Hank's balanced salt solution (HBSS; GIBCO, Santa Clara, Calif.), supplemented with 1 mM CaCl₂, 2 mM MgCl₂, 20 mM HEPES (HBSS; N-2-hydroxyethylpiperazine-N-2-ethanesulfonic acid, pH 7.3) and resuspended in PBS to the desired bacterial concentration. The bacterial concentration was estimated by measuring the A600. An OD600 of 1.0 corresponds to 6×10⁸ viable bacteria, as established from the number of CFU resulting from plating dilutions of bacterial suspension onto L-agar plates and overnight incubation.

Bacterial and MDI-P Protocol

All mice were housed in pathogen-free facilities. Female mice (6-8 weeks of age, The Jackson Laboratory, Bar Harbor, Me.) received an i.p. injection of 100 μg OVA (0.2 ml of 500 μg/ml in normal saline) complexed with alum on days 0 and 14. On days 14, 25, 26, and 27, the mice underwent anesthesia with 0.2 ml i.p. of ketamine (0.44 mg/ml)/xylazine (6.3 mg/ml) in normal saline before receiving a 0.05 ml i.n. dose of either 100 μg OVA in saline (day 14) or 50 μg OVA in saline (days 25, 26, and 27). Control animals received i.p. saline with alum on days 0 and 24 and i.n. saline without alum on days 14, 25, 26, and 27. On day 28, 24 hours after the last i.n. challenge with OVA or saline, the respective OVA-treated and saline control mice received inocula of 1×10⁶ of P. aeruginosa by i.n. delivery for 30 minutes. After 30 minutes of inoculation, either saline, OVA, or 100 μl of MDI-P was then administered i.n. into the lung. To determine bacterial clearance and proliferation, the mice were sacrificed at 0, 4, 24, and 48 hours after P. aeruginosa administration. The right lungs were collected and then homogenized for quantitation of bacterial numbers. The left lungs were obtained for histopathology.

Drug Treatment

MDI-P 100 was produced the day before use. 0.9% sodium chloride, USP (Baxter Healthcare Co, Deerfield, Ill.) was electrolyzed and the pH was adjusted to 7.4 using 2N HCl. Chlorine and hydrogen peroxide content was tested and recorded. In the experiment, OVA-treated mice were i.n. challenged with MDI-P100 and bacterial inoculation after 0, 4, 24, and 48 hours.

TABLE 2

| Time (hr) | SALINE | OVA | MDI-P100 |
|---|---|---|---|
| 0 | n = 4 | n = 4 | n = 4 |
| 4 | 4 | 4 | 4 |
| 24 | 4 | 4 | 4 |
| 48 | 4 | 4 | 4 |

Lung Histology

The upper and lower lobes of the left lung were removed and fixed for 15 hours at 4° C. in Carnoy's solution. The tissues were embedded in paraffin and cut into 5 μm sections before staining with Discombe's solution and counterstaining with methylene blue. Airway infiltration by neutrophils and other inflammatory cells was quantitated by morphometry as previously described. Henderson, W. R., et al., J. Exp. Med., 184:1483-1494 (1996). Airway mucus was identified by the following histochemical procedures: mucin by mucicarmine staining, acidic sulfated mucosubstances, hyaluronic acid, and sialomucins by alcian blue, pH 2.5 staining, neutral and acidic mucosubstances by alcian blue, pH 2.5 and periodic acid-Schiff reaction (PAS), and sialic acid-rich, nonsulfated mucosubstances by toluidine blue, pH 4.5 staining as previously described. Id. For localization of bacteria, sections were stained by the special Gram-negative bacteria stain technique. The lungs were excised and inflated with 10% formalin after infection with P. aeruginosa for 4, 24, or 48 h. Parasagittal sections through the fixed lungs were embedded in paraffin, sectioned at 5 μm thickness and stained with hematoxylin and eosin. Morphometry also was done.

Light Microscopic Morphometry

Tissues were obtained and fixed in 10% neutral formaldehyde solution at room temperature overnight. After being embedded in paraffin, the tissues were cut into 5 μm sections. For light microscopy and morphometry, the lung sections were stained with hemotoxylin and eosin to assess the inflammatory cell infiltrate (0-4+scale); 0.05% aqueous eosin with methylene blue counterstaining to identify eosinophils per unit airway (2,200 μm²); Masson's trichrome to determine collagen deposition in the lungs; and alcian blue, pH 2.5, with nuclear fast red counterstaining to identify airway goblet cells (as percent of total airway cells) and the degree of mucus plugging of the airways (0.5-0.8 mm in diameter) with the percent occlusion of airway diameter by mucus classified on 0-4+scale. Morphometry was performed by individuals blinded to the protocol design. Cell counts were determined using the Point Counting Stereology System II software. A minimum of 10 fields throughout the upper and lower left lung tissue are randomly examined for the morphometric analysis.

Results

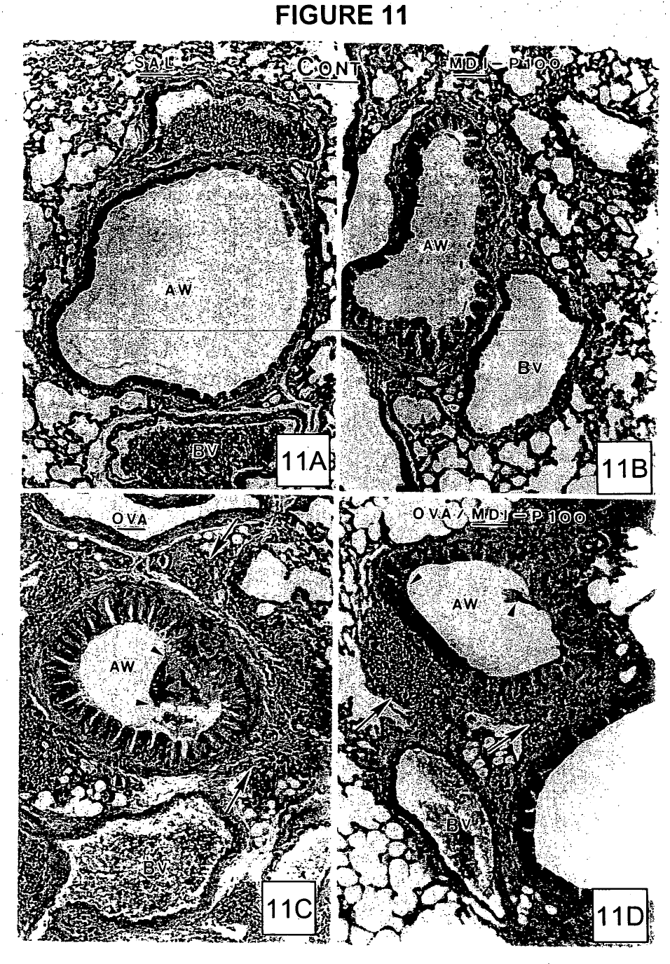

A control group of animals shows the representative expression of the characteristics of pulmonary structure in normal, unchallenged animals. FIG. 11A shows mouse lung after treatment with saline only. The lung has a normal appearance. The airways (AW) were very clean and clear of mucus material. The blood vessel (BV) had a normal appearance without cellular infiltration. FIG. 11B shows mouse lung after treatment with MDI-P100 only. The lung was normal and no abnormal ill-effects were seen on the airway and blood vessel. In contrast, FIG. 11C shows an OVA immunized/challenged mouse lung with typical characteristics of the asthmatic features in the airway (AW), which was filled with mucus and inflammatory epithelial cells. In addition, the airway and blood vessel interstitial areas were infiltrated. In FIG. 11D, lung from an OVA immunized/challenged mouse lung after MDI-P100 treatment shows the airway (AW) contains much less mucus (arrowheads). Cellular infiltration is also reduced (arrows).

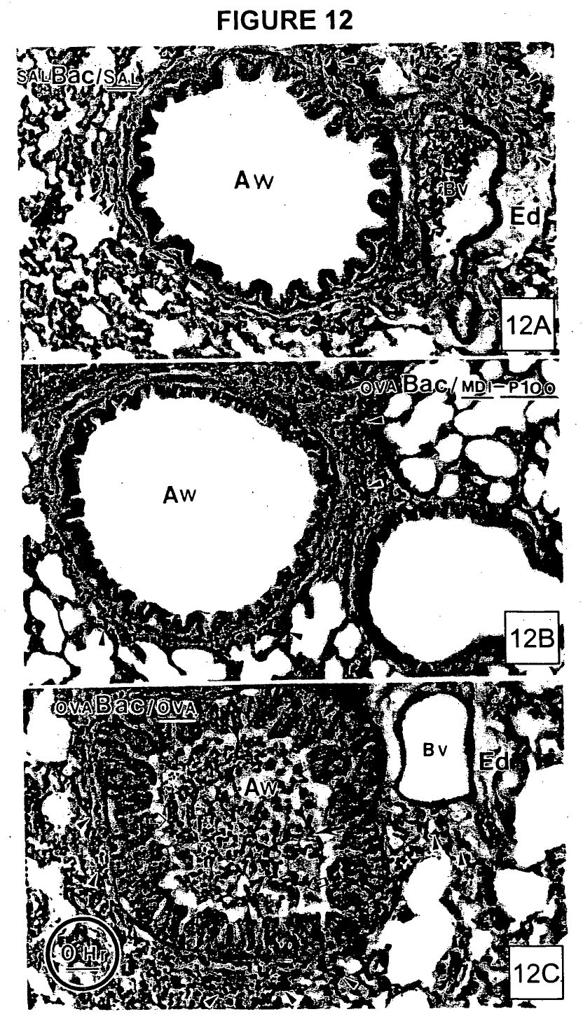

Animals received 1×10⁶ Pseudomonas aeruginosa intranasally for 30 minutes. The animals were sacrificed and the lungs were examined histologically. Saline treated animal airways were clear with little cellular infiltration around the interstitium (arrowheads). Edema (Ed) was found in the tissue around the blood vessel (BV). Inflammatory cells were also seen in the edematic regions (arrows). (FIG. 12A). MDI-P100 treated OVA immunized/challenged lung shows that the airway (AW) is clear without mucus and few cellular infiltrations (arrowheads). (FIG. 12B). OVA immunized/challenged lung without MDI-P100 treatment predominantly showed mucus secretion and cellular infiltration (arrows). Many epithelial cells were observed in the airway (open arrows). Edema (Ed) was observed in the interstitium of the blood vessels (BV). Inflammatory cells were also observed (arrowheads). (FIG. 12C).

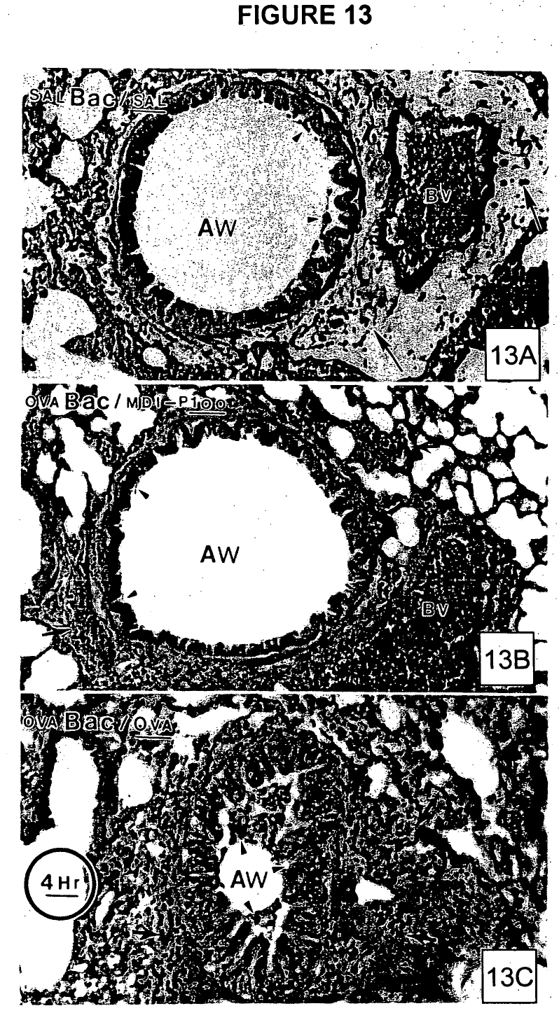

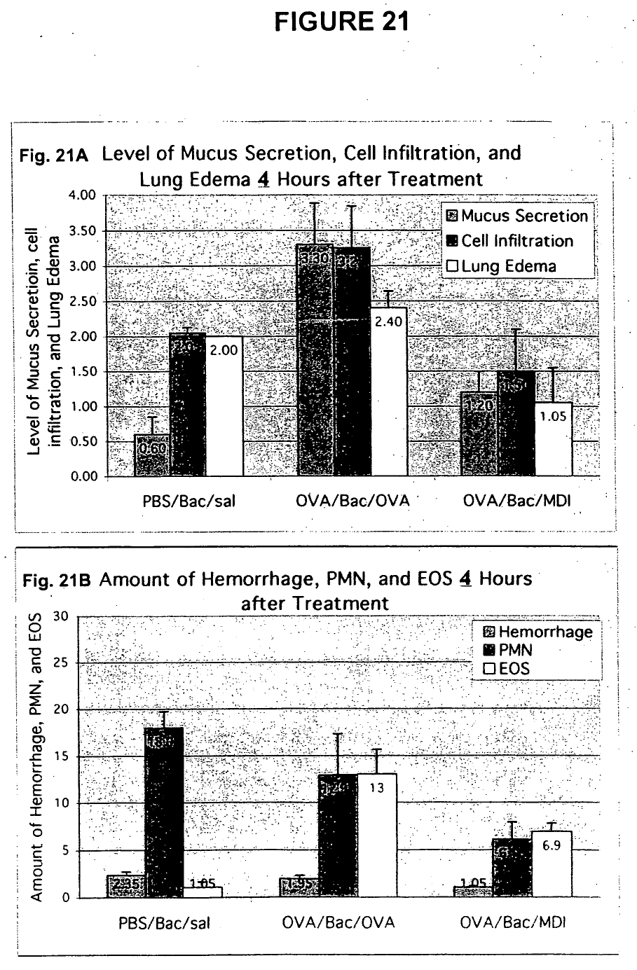

After 4 hours of bacteria incubation, saline-treated lungs showed that P. aeruginosa inoculation induced profound edema in the surrounding blood vessels. Inflammatory cells appeared in the airway (arrowheads) and the interstitial region of the blood vessels (arrows). (FIG. 13A). In MDI-P100 treated lungs, the airways were very clear (arrowheads). In this animal, the OVA-induced cell infiltration is still observed (arrows). (FIG. 13B). In the OVA-treated lungs, bacterial infection produced profound cellular infiltration in mucus-filled lungs (arrowheads) and inflammatory cells were present in airway interstitial area (arrows) and alveoli. (FIG. 13C). A tissue array was present. (FIGS. 21A and 21B).

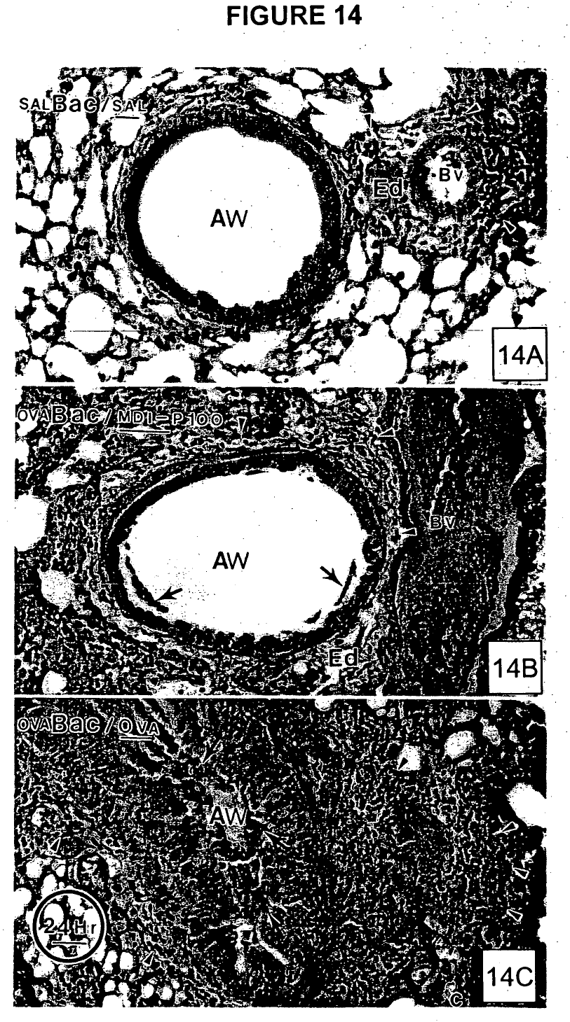

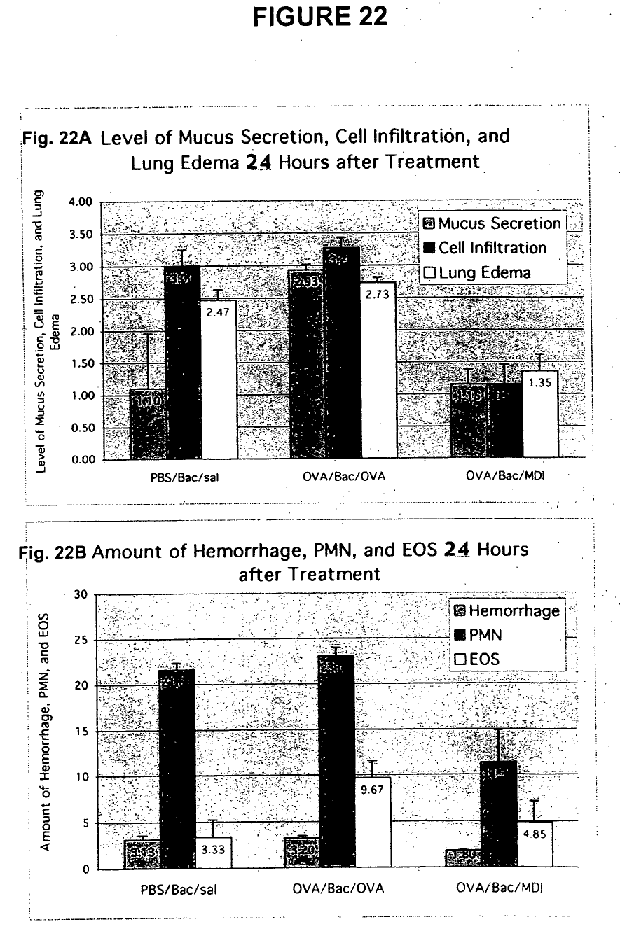

Twenty-four hours after P. aeruginosa inoculation, some of the mouse lung airways in the saline-treated group appeared with only a few inflammatory cells, but the blood vessels were edematous (Ed) and had vasculitis (arrowheads). Neutrophils and edema were also observed in alveoli (arrows). (FIG. 14A). OVA immunized/challeged mice inoculated with bacteria and treated with MDI-P100 after 24 hours, showed the lung airway (AW) contained much less mucus and few inflammatory cells in the airway (arrows) and in the interstitial region (arrowheads). The edema (Ed) is much less obvious. (FIG. 14B). OVA-treated and immunized mouse lungs without MDI-P100 treatment showed severe airway (AW) constriction and mucus production (arrows). Bacteria were observed in the airway, which was loaded with mucus. Cellular infiltration was predominant (arrowheads). A significant number of inflammatory cells were present in this group than the MDI-P treated group (p=0.0025). A comparative analysis of morphometry in the 24 hours after treatment was also shown. (FIGS. 22A and 22B).

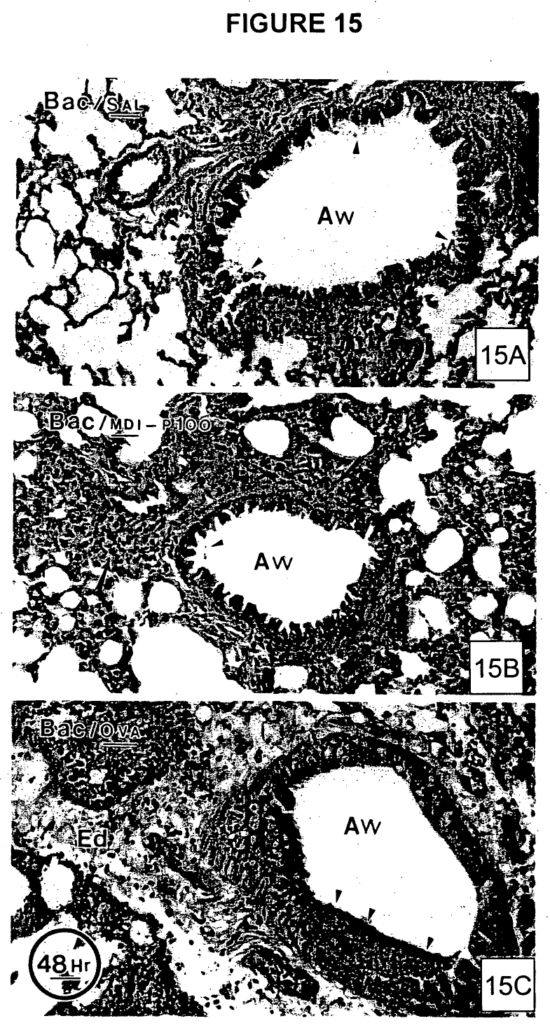

Forty-eight hours after P. aeruginosa i.n. inoculation, animals receiving different treatments showed significant difference in recovery and sickness. The saline treated group of mice showed mucociliary clearance and recovery and expressed few signs of sickness. The histology of the lungs was fairly normal. The airways (AW) were clear, with occasional cellular debris observed (arrowheads). (FIG. 15A). In MDI-P100-treated mice, less severe lung injury occurred than with OVA/OVA-treated mice. The airways were clear with occasional cellular infiltration (arrows). The alveoli were shown with focal edema and hemorrhage (arrows). (FIG. 15B). In OVA/OVA-treated mice, bacterial infection was observed in various stages of disease. The histology of lungs were edematous (Ed) and showed severe hemorrhaging. The airways (AW) were filled with inflammatory cells and epithelial cells were rendered deleterious (FIG. 15C).

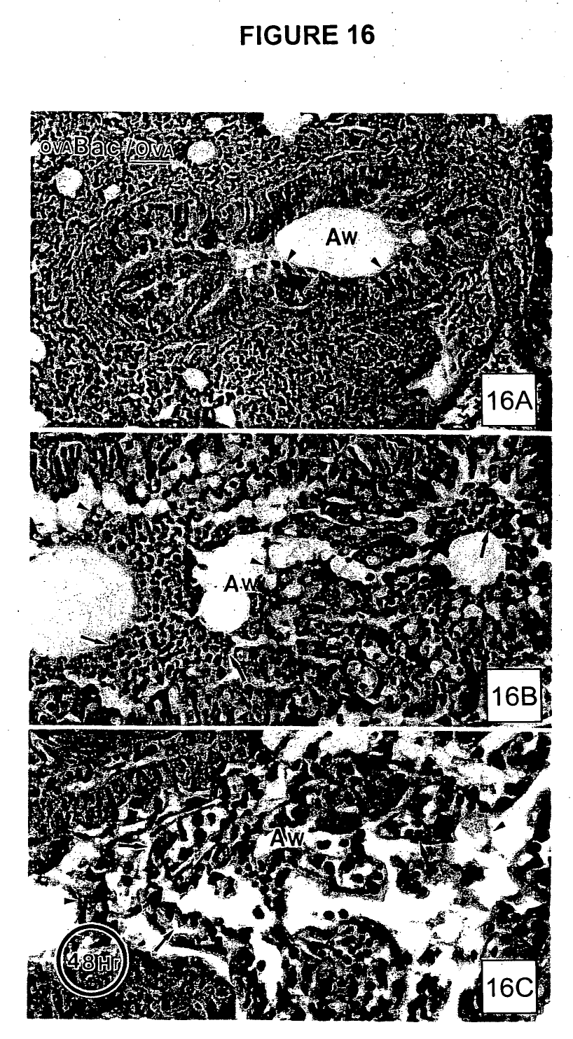

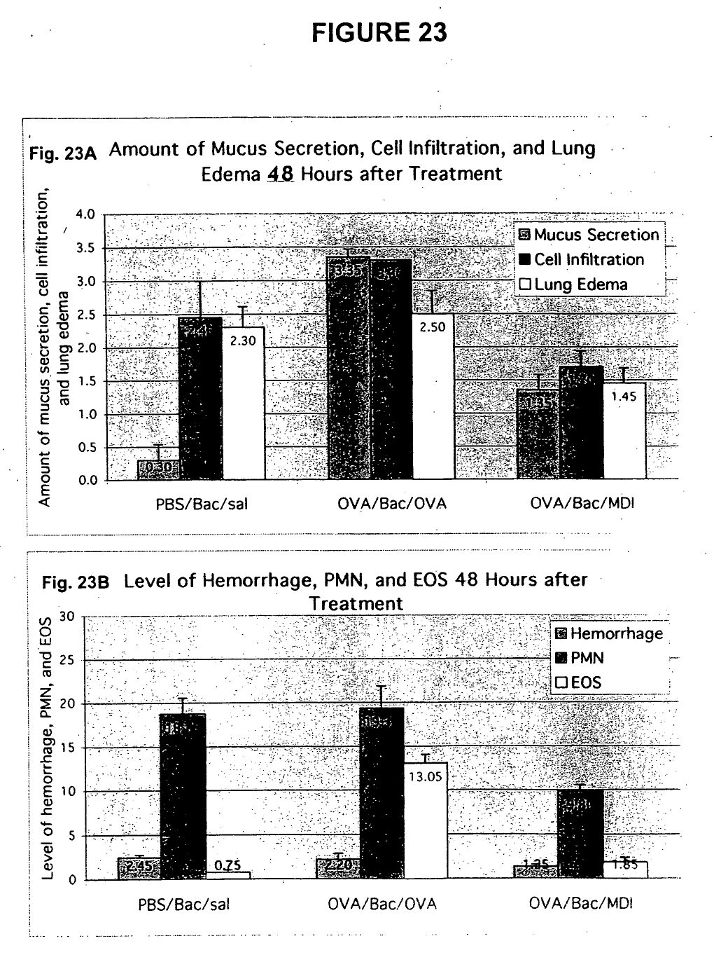

A group of micrographs were selected to illustrate the lung injury induced by P. aeruginosa after 48 hours incubation in OVA immunized/challenged mice. This group of animals exhibit mucus filled lungs, and mimic the pathology of human cystic fibrosis lung disease. OVA immunized/challenged mouse lungs inoculated with bacteria showed the lungs to be severely constricted with mucus blocking the airways (AW). (FIG. 16A). At high magnification (X180), the micrograph clearly shows that the airways (AW) were filled with mucus (arrowheads) and many neutrophils were observed in the airway lumen (arrows). (FIG. 16B). The airways (AW) of the OVA immunized/challenged mouse lungs show the destruction of the epithelia (arrows) and the presence of inflammatory cells in the mucus (arrowheads). The lungs were severely constricted and filled with mucus, and epithelia slashed off the basement membrane was frequently seen (FIG. 16A, FIG. 16B, FIG. 16C, FIGS. 23A and 23B).

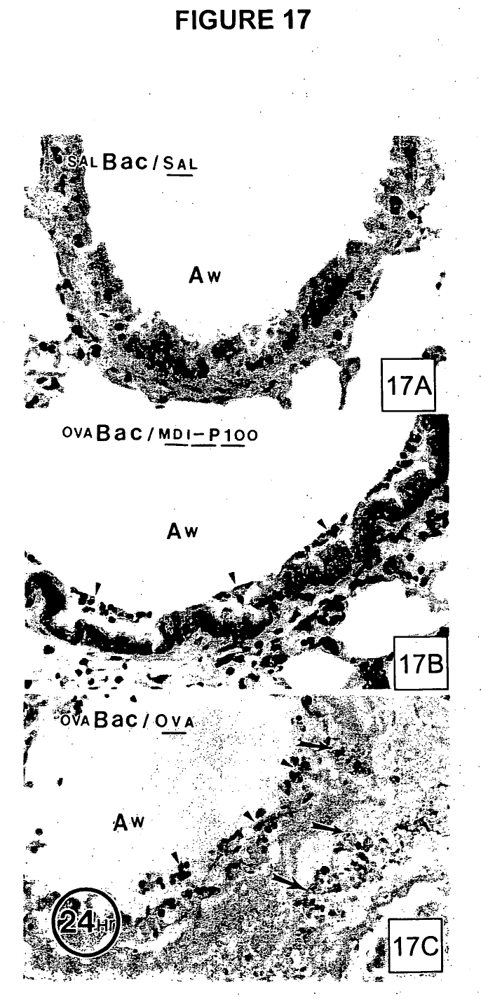

Bacterial localization in lung was shown in different conditions using special staining techniques for gram-negative bacteria. Lung tissues were stained in aniline crystal violet and Gram's Iodine and were counter-stained with neutral red-fast green stains. The gram-negative bacteria P. aeruginosa was stained pink, cell nuclei was stained red, cytoplasm was stained light green, and the interstitium of blood vessels and airway was stained blue. (Culling, CFA 1974, handbook of Histopathological and Histochemical Techniques, Butterworths, London). After 24 hours, only the OVA immunized/challenged only group mice were observed with bacteria localized in lung tissue (FIG. 17C). In the saline-treated mouse lung, no bacteria were observed in the airway and only a few bacteria were observed in the phagocytic vacuoles of macrophages or neutrophils. (FIG. 17A). MDI-P100-treated mouse lungs showed inflammatory cells in the airway (AW) with very little bacteria observed. OVA immunized/challenged mouse lungs showed there was bacteria located around the airway (FIG. 17C).

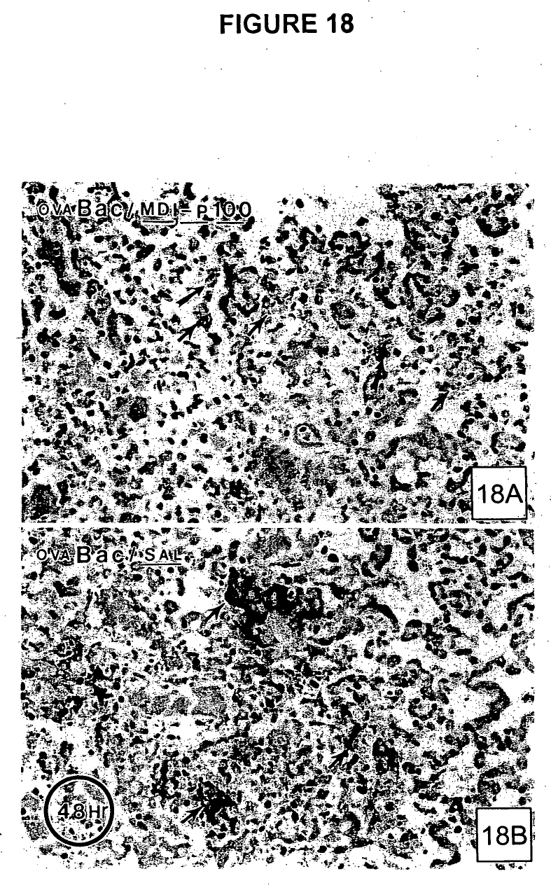

The mucus overproduction mouse model was very useful in studying the P. aeruginosa for in vivo survival and infection of infected mice. The murine model mimics the human disease of cystic fibrosis, expressing characteristics of airway infection and inflammation. After treatment with MDI-P100 for 48 hours, the infection was predominantly located in the alveoli as observed by special negative bacteria stains (FIG. 18A). In the MDI-P-treated animal lungs, the bacteria were found in the large-sized macrophage cytoplasm (arrows) (FIG. 18A), but in the OVA/OVA-treated lungs, a dense positive bacteria stain was observed in the alveoli (arrows) that indicated that a predominant bacterial infection persisted in the lower part of the lungs. (FIG. 18B).

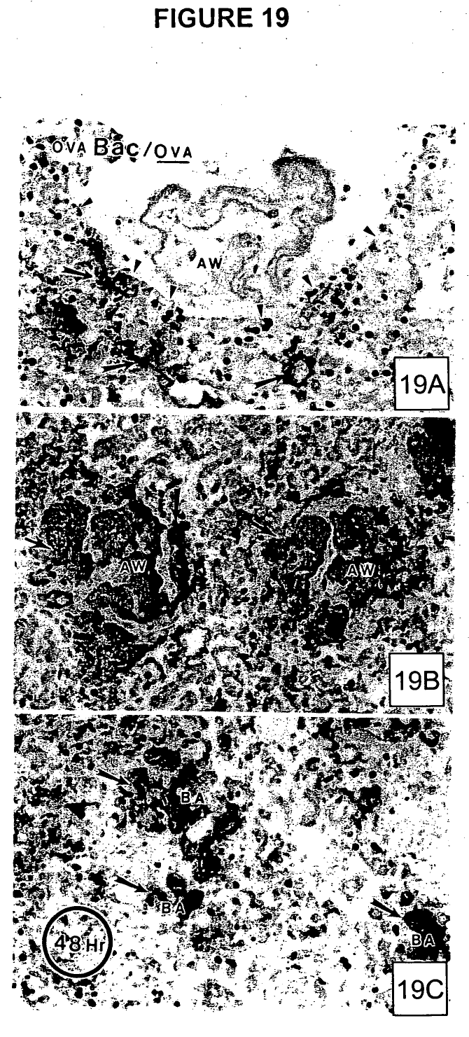

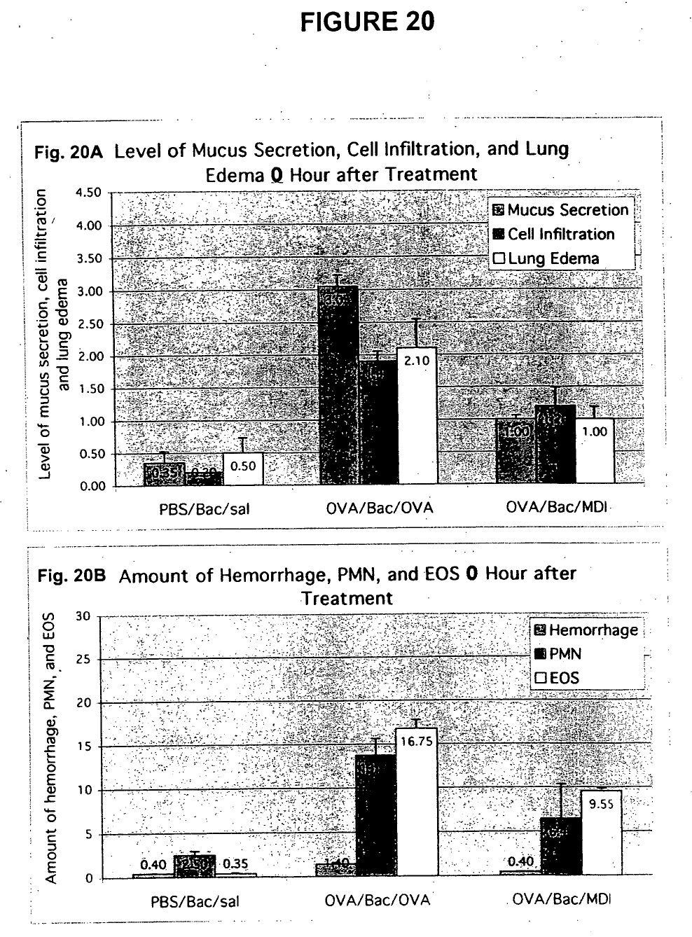

It was evident that in the mucus overproduction OVA-treated mouse lungs inoculated with the P. aeruginosa after 48 hours showed a severe infection in the lung. The most infected areas of the lungs were the airways. Large airways showed the epithelial layer was dense (arrowheads). The infected sites were located around the airway (arrows). (FIG. 19A). Most of the infected sites were around the airways, the small airways (arrows) (FIG. 19B) and in the Broncho-alveolar regions (arrows). (FIG. 19C).

This example (N=48) discloses that MDI-P (100% solution strength) is a useful agent to reduce primary measures of disease in CF, including bacterial P. aeruginosa infection, mucus secretion, lung edema, lung hemorrhage, and lung infiltration by PMN and eosinophils. These findings were established in a new mucus overproduction mouse model designed to closely mimic the CF disease condition found in humans. This mouse model starts with OVA-induced chronic asthmatic mice, which are then infected intranasally with P. aeruginosa to establish a lung disease state comparable with CF patients. Almost all CF patients evidence P. aeruginosa colonization at some time during the disease process, associated with progressive deterioration of lung function in CF. In this model, the airway is filled with mucus occlusion and airway infection following inoculation with P. aeruginosa.

This example demonstrated that MDI-P inhibited P. aeruginosa growth and colonization in the mouse model with airway mucus hypersecretion. Forty-eight hours after treatment, MDI-P treated CF-like mouse lung evidenced a 60% reduction in mucus secretion; a 49% reduction in cytokine chemokine cellular infiltration and a 42% reduction in lung edema, as contrasted with untreated, CF-like condition induced mice. In MDI-P treated mice, the associated level of lung hemorrhage was reduced by 39%, the level of PMN lung infiltration was reduced by 49%, and eosinophil lung infiltration was reduced by 86%, as contrasted with untreated, CF-like condition induced mice. No overt signs of toxicity were found in the primary organs (lungs, liver, spleen, kidneys) of mice treated with MDI-P.

Murine models of lung inflammation are currently most helpful for understanding the role of critical mediators in lung inflammation. In vivo mouse models of asthma have been established which replicate key morphologic and physiologic features of human disease. Ovalbumin (OVA) was used in this example as a model allergen to induce late-phase lung inflammation in normal BALB/c and C57BL/6 mice. Surprisingly, this example demonstrates that the OVA-sensitized/challenged mice can be infected with P. aeruginosa, due to the overproduction of the mucus in their respiratory tract. This model of overproduction of mucus can be useful to elucidate key virulence factors of P. aeruginosa important in lung infection. Use of this murine CF model can also assist in developing new strategies for using MDI-P to prevent and/or control P. aeruginosa infection in patients with cystic fibrosis.

P. aeruginosa-associated chronic airway infection is related to excessive inflammation in cystic fibrosis. P. aeruginosa colonizes the lungs of almost all CF patients and establishes a chronic bronchopulmonary infection. Pukhalsky, supra. A very similar result has been obtained in this example, with resulting airway and bronchopulmonary infection in this mouse model paralleling the morphology of CF in humans. Chronic P. aeruginosa lung infection in CF is characterized by the continued massive influx of PMN as we have seen in this study. PMN are an essential component in chronic P. aeruginosa lung inflammation in CF and have been extensively studied, but the mechanism of excessive PMN-infiltration remains poorly understood. Sapru, K., et al., Clin. Exp. Immunol., 115:103-9 (1999).

Important in this inflammatory process in the lungs of patients with CF is the large set of virulence factors produced by the bacterial infection. The initial interaction of bacteria with the host involves a major change in the expression of many of the bacterial genes to facilitate survival of the organisms. Further, the infection with P. aeruginosa progresses from an initial colonization phase to a chronic infection, which is characterized by conversion of the bacteria to mucoidy variants.

A unique feature of this murine model of allergic airway inflammation is the development of airway goblet cell hyperplasia and mucus hypersecretion. OVA-immunize/challenged mice have morphologic evidence for widespread mucus plugging of the airways. Whereas <10% of the airway cells are mucus glands in control mice, >80% are mucus-secreting goblet cells in OVA-sensitized/challenged animals. The majority (74%) of the airways of the OVA-treated mice exhibit at least 30% occlusion of the airway lumen by mucus. When the amount of mucus glycoprotein recovered in the BAL fluid is quantitated, a seven-fold increase in airway mucin is demonstrated in OVA-treated mice compared to control mice.

Therefore, this example shows an animal model with CF-like characteristics and demonstrates that MDI-P is an effective agent to treat these characteristics.

All publications, patents, and patent documents cited herein are hereby incorporated by reference in their entirety, as though individually incorporated by reference.

It should be noted that as used herein, the terms “a”, “an”, “and “the” should be construed to cover both single and plural referents unless the context clearly dictates otherwise.

The invention has been described with reference to various specific and preferred embodiments and techniques; however, it should be understood that many variations and modifications can be made while remaining within the spirit and scope of the invention. The use of such embodiments, techniques, examples, and exemplary language is intended only to further illuminate the invention and does not limit the scope of the invention unless otherwise claimed.

We claim:

1. A method for treating or preventing a respiratory disorder in an animal, comprising administering an electrolyzed saline solution to the animal.

2. The method of claim 1, wherein the electrolyzed saline solution comprises ozone and one or more active species selected from the group consisting of: active chlorine species, active oxygen species, and active hydrogen species.

3. The method of claim 2, wherein the active chlorine species comprises at least one of an active chlorine species selected from the group consisting of: free chlorine, hypochlorous acid and hypochlorite ion.

4. The method of claim 1, wherein the solution is prepared by subjecting a 1% or less saline solution to electrolysis under conditions sufficient to produce the desired active ingredients.

5. The method of claim 4, wherein the solution is prepared using a saline solution with a starting sodium chloride solution selected from the group consisting of: 0.9% NaCl (w/vol), 0.45% NaCl (w/vol), and 0.215% NaCl (wt/vol).

6. The method of claim 1, wherein the solution comprises HOCl⁻¹, OCL⁻¹, Cl⁻¹, Cl₂, O₂³, O₃, and H₂O₂.

7. The method of claim 1, wherein the solution is administered intranasally or by inhalation.

8. The method of claim 1, wherein the solution is administered intranasally or by inhalation to a mammal at a dosage from about 0.25 ml/kg/day body weight to about 4 ml/kg/day body weight.

9. The method of claim 8, wherein the mammal is a human.

10. The method of claim 1, wherein the respiratory disorder is asthma.

11. A method for treating or preventing a respiratory disorder in a animal, comprising administering an electrolyzed saline solution to the animal, wherein the electrolyzed saline solution comprises from about 0.1 ppm to about 100 ppm ozone and one or more active species selected from the group consisting of: about 5 ppm to about 300 ppm of at least one active chlorine species, about 0.1 ppm to about 300 ppm of at least one active oxygen species, about 5 ppm to about 300 ppm of at least one active hydrogen species, and combinations thereof.

12. The method of claim 11, wherein the active chlorine species comprises at least one of an active chlorine species selected from the group consisting of: free chlorine, hypochlorous acid and hypochlorite ion.

13. The method of claim 11, wherein the solution comprises about 55 ppm to about 80 ppm of at least one active chlorine species.

14. The method of claim 13, wherein the solution further comoprises less than about 15 ppm hydrogen peroxide.

15. The method of claim 11, wherein the solution comprises HOCl⁻¹, OCL⁻¹, Cl⁻¹, Cl₂, O₂³, O₃, and H₂O₂.

16. The method of claim 11, wherein the solution is administered intranasally or by inhalation.

17. The method of claim 11, wherein the solution is administered intranasally or by inhalation to a mammal at a dosage from about 0.25 ml/kg/day body weight to about 4 ml/kg/day body weight.

18. The method of claim 17, wherein the mammal is a human.

19. The method of claim 18, wherein the respiratory disorder is asthma.

20. A method for treating or preventing airway inflammation in an animal, comprising administering an electrolyzed saline solution to the animal.

21. The method of claim 20, wherein the electrolyzed saline solution comprises ozone and one or more active species selected from the group consisting of: active chlorine species, active oxygen species, and active hydrogen species.

22. The method of claim 21, wherein the active chlorine species comprises at least one of an active chlorine species selected from the group consisting of: free chlorine, hypochlorous acid and hypochlorite ion.

23. The method of claim 20, wherein the solution is prepared by subjecting a 1% or less saline solution to electrolysis under conditions sufficient to produce the desired active ingredients.

24. The method of claim 23, wherein the solution is prepared using a saline solution with a starting sodium chloride solution selected from the group consisting of: 0.9% NaCl (w/vol), 0.45% NaCl (w/vol), and 0.215% NaCl (wt/vol).

25. The method of claim 20, wherein the solution comprises HOCl⁻¹, OCL⁻¹, Cl⁻¹, Cl₂, O₂³, O₃, and H₂O₂.

26. The method of claim 20, wherein the solution is administered intranasally or by inhalation.

27. The method of claim 20, wherein the solution is administered intranasally or by inhalation to a mammal at a dosage from about 0.25 ml/kg/day body weight to about 4 ml/kg/day body weight.

28. The method of claim 27, wherein the mammal is a human.

29. The method of claim 20, wherein the airway inflammation is asthma-related.

30. A method for treating or preventing airway inflammation in a animal, comprising administering an electrolyzed saline solution to the animal, wherein the electrolyzed saline solution comprises from about 0.1 ppm to about 100 ppm ozone and one or more active species selected from the group consisting of: about 5 ppm to about 300 ppm of at least one active chlorine species, about 0.1 ppm to about 300 ppm of at least one active oxygen species, about 5 ppm to about 300 ppm of at least one active hydrogen species, and combinations thereof.

31. The method of claim 30, wherein the active chlorine species comprises at least one of an active chlorine species selected from the group consisting of: free chlorine, hypochlorous acid and hypochlorite ion.

32. The method of claim 30, wherein the solution comprises about 55 ppm to about 80 ppm of at least one active chlorine species.

33. The method of claim 32, wherein the solution further comprises less than about 15 ppm hydrogen peroxide.

34. The method of claim 30, wherein the solution comprises HOCl⁻¹, OCL⁻¹, Cl⁻¹, Cl₂, O₂³, O₃, and H₂O₂.

35. The method of claim 30, wherein the solution is administered intranasally or by inhalation.

36. The method of claim 30, wherein the solution is administered intranasally or by inhalation to a mammal at a dosage from about 0.25 ml/kg/day body weight to about 4 ml/kg/day body weight.

37. The method of claim 36, wherein the mammal is a human.

38. The method of claim 30, wherein the airway inflammation is asthma-related.

39. A method for treating cystic fibrosis in an animal, comprising administering an electrolyzed saline solution to the animal.

40. The method of claim 39, wherein the electrolyzed saline solution comprises ozone and one or more active species selected from the group consisting of: active chlorine species, active oxygen species, and active hydrogen species.

41. The method of claim 40, wherein the active chlorine species comprises at least one of an active chlorine species selected from the group consisting of: free chlorine, hypochlorous acid and hypochlorite ion.

42. The method of claim 39, wherein the solution is prepared by subjecting a 1% or less saline solution to electrolysis under conditions sufficient to produce the desired active ingredients.

43. The method of claim 42, wherein the solution is prepared using a saline solution with a starting sodium chloride solution selected from the group consisting of: 0.9% NaCl (w/vol), 0.45% NaCl (w/vol), and 0.215% NaCl (wt/vol).

44. The method of claim 39, wherein the solution comprises HOCl⁻¹, OCL⁻¹, Cl⁻¹, Cl₂, O₂³, O₃, and H₂O₂.

45. The method of claim 39, wherein the solution is administered intranasally or by inhalation.

46. The method of claim 39, wherein the solution is administered intranasally or by inhalation to a mammal at a dosage from about 0.25 ml/kg/day body weight to about 4 ml/kg/day body weight.

47. The method of claim 46, wherein the mammal is a human.

48. A method for treating cystic fibrosis in a animal, comprising administering an electrolyzed saline solution to the animal, wherein the electrolyzed saline solution comprises from about 0.1 ppm to about 100 ppm ozone and one or more active species selected from the group consisting of: about 5 ppm to about 300 ppm of at least one active chlorine species, about 0.1 ppm to about 300 ppm of at least one active oxygen species, about 5 ppm to about 300 ppm of at least one active hydrogen species, and combinations thereof.

49. The method of claim 48, wherein the active chlorine species comprises at least one of an active chlorine species selected from the group consisting of: free chlorine, hypochlorous acid and hypochlorite ion.

50. The method of claim 48, wherein the solution comprises about 55 ppm to about 80 ppm of at least one active chlorine species.

51. The method of claim 50, wherein the solution further comprises less than about 15 ppm hydrogen peroxide.

52. The method of claim 48, wherein the solution comprises HOCl⁻¹, OCL⁻¹, Cl⁻¹, Cl₂, O₂³, O₃, and H₂O₂.

53. The method of claim 48, wherein the solution is administered intranasally or by inhalation.

54. The method of claim 48, wherein the solution is administered intranasally or by inhalation to a mammal at a dosage from about 0.25 ml/kg/day body weight to about 4 ml/kg/day body weight.

55. The method of claim 54, wherein the mammal is a human.

56. A methods for inducing a cystic fibrosis-like syndrome in a mouse comprising:
a) inducing chronic asthma in a mouse and
b) administering bacteria to the mouse of step a) such that the mouse develops a cystic fibrosis-like syndrome.

57. The method of claim 56, wherein the chronic asthma of step a) is induced by an allergen immunization/challenge protocol.

58. The method of claim 58, wherein the allergen immunization/challenge protocol comprises administration of ovalbumin to the mouse.

59. The method of claim 58, wherein the bacteria comprises Pseudomonas aeruginosa.

60. The method of claim 59, wherein the Pseudomonas aeruginosa comprises strain CF 18.

61. The method of claim 56, wherein the cystic fibrosis-like syndrome comprises one or more characteristics of human cystic fibrosis selected from the group consisting of: bacterial infection, mucus secretion, lung edema, lung hemorrhage, and lung infiltration by polymorphonuclear leukocytes and eosinophils.

62. A method for inducing a cystic fibrosis-like syndrome in a mouse comprising:
a) administering ovalbumin to a mouse such that a chronic asthma condition develops in the mouse and
b) administering Pseudomonas aeruginosa to the mouse of step a) such that the mouse develops a cystic fibrosis-like syndrome.

63. The method of claim 62, wherein the ovalbumin is administered on one or more of days 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, and/or 31 in a month.

64. The method of claim 62, wherein the ovalbumin is administered intraperitoneally, intranasally, or a combination of intraperitoneally and intranasally.

65. The method of claim 62, wherein the ovalbumin is administered in an amount of about 15 mg, about 20 mg, about 25 mg, about 30 mg, about 40 mg, about 50 mg, about 60 mg, about 70 mg, about 80 mg, about 90 mg, about 100 mg, about 125 mg, about 150 mg, about 175 mg, about 180 mg, or more per dose.

66. The method of claim 65, wherein the ovalbumin is complexed with alum before administration.

67. The method of claim 62, wherein the Pseudomonas aeruginosa is administered intraperitoneally, intranasally, or a combination of intraperitoneally and intranasally.

68. The method of claim 62, wherein the Pseudomonas aeruginosa is administered in an amount of about 1×10³, about 1×10⁴, about 1×10⁵, about 1×10⁶, about 1×10⁷, about 1×10⁸, or more per dose.

69. A method of screening pharmaceutical agents for effectiveness in the treatment of cystic fibrosis comprising:
a) inducing chronic asthma in a mouse;
b) administering bacteria to the mouse of step a) such that the mouse develops a cystic fibrosis-like syndrome;
c) treating the mouse with the pharmaceutical agent of interest; and
d) evaluating the characteristics of the cystic fibrosis-like syndrome to determine if there is a difference in the characteristics of the syndrome after treatment with the pharmaceutical compared with the characteristics of the syndrome prior to treatment with the pharmaceutical;
wherein the effectiveness of the pharmaceutical agent in the treatment of cystic fibrosis is demonstrated by a positive change in the characteristics of the syndrome after treatment with the pharmaceutical compared to the characteristics of the syndrome prior to treatment.

70. The method of claim 69, wherein a positive change in the characteristics or pathology of the syndrome comprises an improvement in the disorder.

71. The method of claim 69, wherein the chronic asthma is induced by an allergen immunization/challenge protocol.

72. The method of claim 71, wherein the allergen immunization/challenge protocol comprises administration of ovalbumin to the mouse.

73. The method of claim 69, wherein the bacteria comprises Pseudomonas aeruginosa.

74. The method of claim 73, wherein the Pseudomonas aeruginosa comprises strain CF 18.

75. The method of claim 69, wherein the cystic fibrosis-like syndrome comprises one or more characteristics of human cystic fibrosis selected from the group consisting of: bacterial infection, mucus secretion, lung edema, lung hemorrhage, and lung infiltration by polymorphonuclear leukocytes (PMNs) and eosinophils.
